(19) United States
(12) Patent Application Publication
(10) Pub. No.: US 2024/0165162 A1
(43) Pub. Date: May 23, 2024

(54) METHODS FOR ACTIVATION AND EXPANSION OF NATURAL KILLER CELLS AND COMBINATIONS WITH BISPECIFIC ANTIBODIES

(71) Applicant: BOARD OF REGENTS, THE UNIVERSITY OF TEXAS SYSTEM, Austin, TX (US)

(72) Inventors: Katy REZVANI, Houston, TX (US); Elizabeth SHPALL, Houston, TX (US); David MARIN COSTA, Houston, TX (US); Rafet BASAR, Houston, TX (US)

(21) Appl. No.: 18/550,484

(22) PCT Filed: Apr. 7, 2022
(86) PCT No.: PCT/US2022/023920

§ 371 Date: Sep. 14, 2023

Related U.S. Application Data
(60) Provisional application No. 63/228,991, filed on Aug. 3, 2021.
(60) Provisional application No. 63/172,402, filed on Apr. 8, 2021.

(51) Int. Cl. A61K 35/17; A61K 39/00; A61P 35/00; C07K 16/28; C12N 5/0783

(52) U.S. Cl. CPC A61K 35/17; A61P 35/00; C07K 16/28; C12N 5/0646; A61K 2039/505; C12N 2501/2302; C12N 2501/2304; C12N 2501/2307; C12N 2501/2312; C12N 2501/2315; C12N 2501/2318; C12N 2501/2321; C12N 2501/2323

(57) ABSTRACT

Embodiments of the disclosure concern methods and compositions related to preparation and use of combinatorial immunotherapies. In specific embodiments, compositions comprising NK cells prepared in a particular manner also include certain antibodies. These compositions are utilized for treatment, such as for cancer treatment. In particular embodiments, the compositions include complexes of the NK cells and the antibodies in which the antibody is bound to the NK cells and may also bind to another antigen, such as on a cancer cell.

This application claims priority to U.S. Provisional Patent Application Ser. No. 63/172,402, filed Apr. 8, 2021, and also claims priority to U.S. Provisional Patent Application Ser. No. 63/228,991, filed Aug. 3, 2021, both of which applications are incorporated by reference herein in their entirety.

### TECHNICAL FIELD

Embodiments of the disclosure include at least the fields of cell biology, molecular biology, immunology, and medicine, including cancer medicine.

### BACKGROUND

In the field of natural killer (NK) cells as a therapy, certain barriers impede their use, including the requirement for ex vivo expansion because of limited numbers, in addition to their insufficient persistence that impacts their efficacy as an anti-cancer therapy. Cytokine stimulation constitutes an important signal to enhance the functional competency of NK cells to respond to tumor target cells. Furthermore, pre-activation of NK cells overnight with the combination of IL-18, IL-15, and IL-12 has been shown to generates long-lived, memory-like NK cells with enhanced cytokine production upon re-stimulation (Leong et al., 2014). However, procedures for using pre-activated NK cells from peripheral blood (PB) in the absence of ex vivo expansion require cumbersome procedures to generate sufficient amounts for clinical use. In addition, there are other obstacles related to deficient cancer recognition and poor NK cell activation with NK cells. Thus, there is an unmet need for improved strategies to generate highly functional pre-activated NK cells in sufficient number for therapeutic applications and that are particularly efficient at cancer antigen targeting.

### BRIEF SUMMARY

Embodiments of the disclosure concerns methods and compositions related to immunotherapy for a medical condition. The immunotherapy may be utilized for any medical condition in which targeting of cells in need of destruction is clinically useful and for which there are specific antigens on the targeted cells for which antibodies and/or cells may be specific. In specific embodiments, the immunotherapy comprises, consists of, or consists essentially of adoptive cell therapy and antibodies that are configured to bind to the cells. In particular embodiments, the adoptive cell therapy comprises NK cells and the antibodies may be multispecific, including at least bispecific. Thus, in specific embodiments compositions are utilized for therapy that are combinatorial for both NK cells and antibodies, and the combination may be configured such that the antibody is able to bind a surface antigen on the NK cells and this complex of the combination is therapeutically utilized.

In specific embodiments, the NK cells for the methods and compositions may be prepared in a specific manner prior to use, including prior to combination (of any kind) with the antibodies. In particular embodiments, the NK cells are expanded in a particular manner and optionally are pre-activated in a particular manner. For example, the NK cells may be expanded in the presence of particular antigen presenting cells under particular culture conditions, in addition to the NK cells optionally being exposed to one or more cytokines as a pre-activation step.

In particular embodiments, the efficacy of the compositions comprising complexes of the NK cells and the antibodies is enhanced compared to their separate use. In specific cases, the efficacy of the NK cells and the antibodies is synergistic when used as a complex, although in other cases the efficacy of the NK cells and the antibodies is additive.

Embodiments of the disclosure concern compositions, comprising (1) one or more cord blood-, stem cell—(including iPSC), or peripheral blood-derived natural killer (NK) cells; and (2) one or more one antibody molecules, wherein: (a) the antibody is monospecific, wherein an Fc region of the monospecific antibody binds the NK cell and an antigen binding domain of the monospecific antibody binds a target antigen; or (b) the antibody is multispecific and one or more antigen binding domains of the antibody binds a target antigen and another antigen binding domain or domains of the antibody binds an NK cell surface antigen. The NK cell may or may not be expanded. The NK cell may or may not be pre-activated. In some cases, the multispecific antibody is bispecific, trispecific, or multi-specific. In specific cases, in (a) the composition is further defined as a complex between the NK cell and the monospecific antibody, through binding of the Fc region of the monospecific antibody to the NK cell. In specific cases, in (b) the composition is further defined as a complex between the NK cell and the multispecific antibody, through binding of the antigen binding domain or domains of the multispecific antibody that binds the NK cell surface antigen or antigens. The target antigen may be a stem cell antigen, auto-antigen, or a cancer antigen selected from the group consisting of CD19, CD319 (CS1), ROR1, CD20, CD22, CD70, carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD5, CD123, CD23, CD30, CD38, CD56, CD70, CD38, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, HLA-G, TROP2, CD99, and a combination thereof. The NK cell surface antigen may be CD16, CS1, CD56, NKG2D, NKG2C, or any c-type lectin, a costimulatory molecule such as DNAM, 2B4, CD2, an NCR, or KIR. In some cases for the composition, the source of the cord blood is cord blood from 1 donor or pooled from 2 or more individual cord blood units. The CB may be pooled from 3, 4, 5, 6, 7, or 8 individual cord blood units. The NK cells, which may be CD56+) may be derived from cord blood mononuclear cells, from cord blood hematopoietic stem cells, from iPSCs, from peripheral blood, or from NK cell lines. The composition may be used fresh or was cryopreserved. A source of the NK cells may be a fresh source or cryopreserved repository. In some cases, when the NK cells are sourced from cryopreservation, the NK cells were cryopreserved in a medium comprising at least one cryoprotectant, at least one serum or non-serum alternative to serum. In certain embodiments at least one cytokine and/or at least one growth factor may be added to the cryopreservation media. The cryoprotectant may be dimethyl sulfoxide (DMSO), glycerin, glycerol, hydroxyethol starch, dextran trehalose, or a combination thereof. The non-serum alternative may comprise platelet lysate and/or a blood product lysate or human or animal serum albumin. In some cases, the at least one cytokine (which may be interleukin (IL)-1, IL-2, IL-3, IL-4, IL-6, IL-7, IL-9, IL-10, IL-12, IL-13, IL-15, IL-17, IL-18, IL-21, IL-22, IL-23, interferon, tumor necrosis factor, stem cell factor, FLT3-ligand, APRIL, thrombopoietin, erythropoietin, or a combination thereof) is a natural protein, a recombinant protein, a synthetic protein, or a mixture thereof. The NK cells may be modified by the hand of man in one or more ways, such as by comprising one or more engineered antigen receptors, including chimeric antigen receptors or T cell receptors, or CD16, CD32 and/or CD64 receptor. The NK cells may express a heterologous cytokine, such as IL-2, IL-4, IL-7,IL-12, IL-15, IL-18, IL-21 or IL-23. Additionally or alternative, the NK cells may express a suicide gene. The composition may be comprised in a solution or solid comprising one or more cryoprotectants. The composition may be comprised in a pharmaceutically acceptable carrier.

In one embodiment, there is a method of producing any composition encompassed herein, comprising the steps of (a) optionally expanding NK cells in a culture comprising an effective amount of: (1) a cytokine selected from the group consisting of IL-2, IL-15, and/or IL-21; and (2) antigen presenting cells/feeders or NK activating beads; and (b) providing the antibody molecules to the NK cells, and when expanding providing the antibody molecules to the NK cells before and/or after expanding. In some cases, the method comprises a pre-activating step prior to and/or after the expanding step, wherein the NK cells are pre-activated in a culture comprising an effective concentration of one or two or three or more of IL-2, IL-12, IL-15, and IL-18. In some cases, IL-12 is utilized in lieu of IL-15 in the culture. The providing step may be further defined as culturing the NK cells with the antibody molecules for a specific duration of time (e.g., about 5 minutes to about 24 hours or more) or combining the NK cells and the antibody molecules just prior to infusion. The culture may or may not comprise Plasma-Lyte A and/or human serum albumin. Following culture, the compositions may or may not be infused into a recipient subject without washing first. The NK cells may be depleted of CD3+, CD14+ and/or CD19+ cells. The depleting step may occur prior to the pre-activation step, and/or prior to expansion with feeder cells and/or NK cell-activating beads, and/or prior to culture with one or more cytokines, and/or prior to infusion. In some cases, the method further comprises the step of obtaining the NK cells from cord blood, wherein the cord blood does not comprise cord tissue.

For the production method the antigen presenting cells may be artificial (aAPCs), and may express CD137 ligand. The aAPCs may further express one or more membrane-bound cytokines, such as membrane-bound IL-21 (mIL-21) and/or membrane-bound IL-15 (mIL-15). The aAPCs may have essentially no expression of endogenous HLA class I, II, and/or CD1d molecules. In some cases, the aAPCs express ICAM-1 (CD54) and/or LFA-3 (CD58) and/or CD48. The aAPCs may be further defined as leukemia cell-derived aAPCs, and the leukemia-cell derived aAPCs may be K562 cells, such as engineered to express CD137 ligand and/or mIL-21. In any case, the aAPCs may have been engineered by transfer by a vector of any kind, including viral or non-viral vector, and including retroviral transduction. The aAPCs may or may not be irradiated. The pre-activating step may be for 10-20 hours, 14-18 hours, or for 16 hours. The culture for the pre-activating step may comprise IL-18 and/or IL-15 at a concentration of 1-1000 ng/mL. The culture for the pre-activating step may comprise IL-12 at a concentration of 0.1-1000 ng/mL. The culture for the pre-activating step may comprise IL-12 at a concentration of 1-1000 ng/ml, including of 10 ng/mL. In some cases, the method further comprises washing the pre-activated NK cells prior to and/or after the expanding step, and the washing may be once or may be performed multiple times. When expanding, the expanding may be for 5-60 days, including 12-16 days, such as for 18-24 days. In some matters, the pre-activated NK cells and aAPCs are present in the expansion culture at a ratio of 3:1 to 1:3, including at a ratio 1:2. The expansion culture may or may not further comprise IL-2, such as at a concentration of 10-500 U/mL, including 100-300 U/mL, such as 200 U/mL. In any aspect of the method, the IL-12, IL-18, IL-15, and/or IL-2 may or may not be recombinant. In some cases, the IL-2 is replenished in the expansion culture every 2-3 days. IL-2 or IL-15 or IL-18 or any combination of the cytokines may be added to the expansion culture at least a second time. The APCs may be added to the expansion culture at least a second time. In some cases, one or more steps of the method are performed in serum-free media.

In one embodiment, there is a method of treating a disease or disorder in a subject (including mammalian, such as human), comprising administering a therapeutically effective amount of any composition encompassed herein, and the NK cells of the composition may be autologous or allogeneic with respect to the subject. The disease or disorder may be cancer (hematological cancer or a solid tumor), inflammation, graft versus host disease, transplant rejection, an autoimmune disorder, an immunodeficiency disease, a B cell malignancy, or an infection. Hematological cancers include a leukemia selected from the group consisting of an acute lymphoblastic leukemia (ALL), chronic lymphocytic leukemia (CLL), acute myelogenous leukemia (AML), and a chronic myelogenous leukemia (CML). The disorder may be graft versus host disease (GVHD), multiple sclerosis, inflammatory bowel disease, rheumatoid arthritis, type I diabetes, systemic lupus erythrematosus, contact hypersensitivity, asthma, and/or Sjogren's syndrome.

In some cases, the method further comprises administering at least a second therapeutic agent to the subject, such as a therapeutically effective amount of one or more anti-cancer agents (chemotherapy, radiotherapy, gene therapy, surgery, hormonal therapy, anti-angiogenic therapy and/or immunotherapy, for example), one or more immunomodulatory agents, and/or one or more immunosuppressive agents, such as one or more of a calcineurin inhibitor, an mTOR inhibitor, an antibody, a chemotherapeutic agent irradiation, a chemokine, an interleukins or an inhibitor of a chemokine or an interleukin. Any second agent for any disease may be an antibody including a monoclonal, bispecific, trispecific, or tetraspecific antibody.

In any method, the composition (and/or the at least a second therapeutic agent) may be administered intravenously, intraperitoneally, intratracheally, intratumorally, intramuscularly, endoscopically, intralesionally, percutaneously, subcutaneously, regionally, or by direct injection or perfusion or infusion.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description. It is to be expressly understood, however, that the disclosure is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1. Schmatic diagram detailing one embodiment of an experimental plan for expanding cord blood derived NK cells and preloading them with antibody.

FIG. 2. Margetuximab binds to P+E NK cells with higher affinity compared to normal expanded NK cells.

FIG. 3. Margetuximab-loaded NK cells show enhanced cytotoxicity against HER2+tumor cells in a long-term Xcelligence killing assay.

FIG. 4A-4C. Margetuximab-loaded NK cells lead to enhanced tumor control in an NSG mouse model of HER2+ovarian cancer (SKOV3).

FIG. 5. Amivantamab binds to P+E NK cells with higher affinity compared to normal expanded NK cells.

FIG. 6. Amivantamab-loaded NK cells show enhanced cytotoxicity against EGFR+/cMET+tumor cells in a short term 51Cr release killing assay.

FIGS. 7A-7C. Amivantamab-loaded NK cells show enhanced cytotoxicity against EGFR+/cMET+tumor cells in a long-term Xcelligence killing assay.

FIGS. 8A-8C. Amivantamab-loaded NK cells lead to enhanced tumor control in an NSG mouse model of EGFR+/c-MET+ovarian cancer (SKOV3).

FIG. 9. Imgatuzumab binds to preactivated and expanded (PE) NK cells with higher affinity compared to normal expanded (NE) NK cells.

FIG. 10. Imgatuzumab-loaded NK cells show enhanced cytotoxicity against EGFR+tumor cells in a short term 51Cr release killing assay.

FIGS. 11A-11C. Imgatuzumab-loaded NK cells show enhanced cytotoxicity against EGFR+tumor cells in a long-term Xcelligence killing assay.

FIGS. 12A-12C. Imgatuzumab-loaded NK cells lead to enhanced tumor control in an NSG mouse model of EGFR+ovarian cancer (SKOV3).

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z.” “(x and y) or z,” “x or (y and z),” or “x or y or z.” It is specifically contemplated that x, y, or z may be specifically excluded from an embodiment.

It is contemplated that any embodiment discussed in this specification can be implemented with respect to any method or composition of the invention, and vice versa. Furthermore, compositions of the invention can be used to achieve methods of the invention.

Throughout this application, the term “about” is used according to its plain and ordinary meaning in the area of cell and molecular biology to indicate that a value includes the standard deviation of error for the device or method being employed to determine the value.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In specific embodiments, a vector is engineered through recombinant nucleic acid technologies, and a cell is engineered through transfection or transduction of an engineered vector.

The term “load,” “loaded,” or loading” as used herein refers to adoptive cell therapy cells having one or more antibodies bound to the cells on the surface of the cells.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “pre-activated” or “pre-activation” as used herein refers to exposure of NK cells to IL-12, IL-15 or IL-2, and/or IL-18 that results in increased signaling pathways related to NK cell effector function, such as enrichment of genes involved in the IFN-γ response, TNF signaling, IL-2/STAT5 signaling, IL-6/JAK/STAT3 signaling, mTOR pathway and/or genes related to inflammatory immune responses. In specific cases there is increased expression of TRAIL, NKp44 and/or CD69.

The term “pre-load,” “pre-loaded.” or “pre-loading” as used herein refers to adoptive cell therapy cells that have had one or more antibodies bound to the cells on the surface of the cells prior to use of the cells for any reason.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

The term “subject,” as used herein, generally refers to an individual having a that has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may being undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of one or more symptoms of the disease or condition, or the delaying of the progression of the disease or condition. “Treatment” does not necessarily indicate complete eradication or cure of the disease or condition, or associated symptoms thereof.

I. Natural Killer (NK) Cells and Methods of Production

NK cells are emerging as an exciting source of cellular immunotherapy for patients with malignant hematologic disease as well as solid tumors; however, most studies using adoptively transferred NK cells have been limited by inadequate persistence, poor in vivo expansion and disappointing anti-tumor activity of the infused cells. Thus, a barrier to overcome in the field of NK immunotherapy is the need for biology-driven approaches to increase NK cell antitumor functionality, such as by manipulation of the cells prior to administration as therapy. Accordingly, in certain embodiments, the present disclosure provides methods for production of NK cells having enhanced efficacy of any kind compared to NK cells that are not so manipulated. Some embodiments of the present disclosure concern the isolation, activation, and expansion of NK cells, including for cancer immunotherapy.

In particular embodiments, the disclosure encompasses loaded, pre-activated, (optionally) and expanded (optionally) NK cells produced by the present methods that exhibit enhanced anti-tumor functionality against cancer. Pre-activated and expanded NK cells also display enhanced antibody-dependent cellular cytotoxicity (ADCC), in specific embodiments.

A. NK Cells, Generally

In certain embodiments, NK cells utilized in the methods may be derived from any suitable source, such as cord blood (CB), including human CB. In particular cases, the NK cells are not derived from cord tissue (the insulating material (i.e., the Wharton's jelly) surrounding the vessels of the umbilical cord). In alternative embodiments, the NK cells are derived from human peripheral blood mononuclear cells (PBMC), unstimulated leukapheresis products (PBSC), human embryonic stem cells (hESCs), induced pluripotent stem cells (iPSCs), and/or bone marrow, or NK cells lines derived from patients, such as NK-92, by methods well known in the art. In particular embodiments, the NK cells are isolated from pooled CB. The CB may be pooled from 2, 3, 4, 5, 6, 7, 8, 9, 10, or more units. The NK cells may be autologous or allogeneic with respect to a recipient individual. The isolated NK cells may or may not be haplotype matched for the subject to be administered the cell therapy. NK cells may or may not be detected by specific surface markers, such as CD16 and/or CD56 in humans. In some cases, the NK cells are depleted for the presence of one or more surface markers, such as depleted for CD3+, CD14+ and/or CD19+ cells. In specific embodiments, the NK cells are CD3−CD56+.

In certain aspects, the NK cells are isolated by the previously described method of ex vivo expansion of NK cells (Spanholtz et al., 2011; Shah et al., 2013). In this method, CB mononuclear cells are isolated by ficoll density gradient centrifugation. The cell culture may be depleted of any cells expressing CD3 and may be characterized to determine the percentage of CD56+/CD3 cells or NK cells. In other methods, umbilical CB is used to derive NK cells by the isolation of CD34+ cells.

B. Loading of NK Cells

The NK cells are loaded with antibodies prior to use, in particular embodiments. The NK cells may be loaded in any specific manner, including in culture or immediately before infusion, for example, to produce a complex of NK cells with the antibodies. The conditions are suitable enough to allow for an effective amount of antibody to bind to the surface of the NK cells. In the case of use of monospecific antibodies, the Fc region of the monospecific antibody binds the NK cell while the antigen binding domain of the monospecific antibody is free to bind its target antigen. In the case of use of multispecific antibodies, one or more antigen binding domains of the antibody binds the surface of the NK cells, such as through an antigen on the surface of the NK cells, and the other antigen binding domain is free to bind its target antigen.

The culture conditions by which the NK cells become loaded may or may not be of a particular type having one or more specific parameters. In particular embodiments, the loading of the NK cells occurs in culture at a specific temperature, such as 37° C., although in alternative embodiments the temperature is 36° C. or 38° C., or lower or higher. The duration of the loading step may be for any suitable amount of time, such as in a range of one minute to 24 hours or longer. For example, the range may be in the range of 1 min to 24 hrs, 1 min to 18 hrs, 1 min to 12 hours, 1 min to 6 hrs, 1 min to 1 hr, 30 min to 24 hrs, 30 min to 18 hrs, 30 min to 12 hrs, 30 min to 6 hrs, 30 min to 1 hr, 1-24 hrs, 1-18 hrs, 1-12 hrs, 1-6 hrs, 6-24 hrs, 6-18 hrs, 6-12 hrs, 12-24 hrs, 12-18 hrs, or 18-24 hrs. In specific embodiments, the cell culture media is basal media or complex media. In some cases, the culture comprises one or more reagents that were utilized during pre-activation and/or expansion steps, while in other cases the culture does not. In specific embodiments, the culture comprises one or more cytokines, including one or more of IL-12, IL-15, IL-2, and IL-18, for example. In some embodiments, the culture comprises APCs of any kind.

The antibodies of the compositions are subjected in an effective amount to an effective amount of NK cells of the disclosure, thereby producing a complex that is “chimeric antigen receptor-like.” In particular, an antigen binding domain of the antibody binds to the NK cells, such as through the antigen that is a cell surface protein. A plurality of antibodies may be subjected to a plurality of NK cells such that there are multiple complexes of cell/antibody. The antibodies may be of any time, including monospecific, bispecific, or multispecific, and in specific cases the antibody engages both the NK cell and a target antigen through an antigen binding domain of the antibody (such as with engagers in the art that are fusion proteins consisting of two single-chain variable fragments (scFvs) of different antibodies). In examples wherein the antibody is monospecific, an antigen binding domain of the antibody binds a target antigen, such as a cancer antigen, and another part of the antibody binds the NK cells, such as an Fc region of the antibody. In cases wherein the antibody is multispecific, one or more antigen binding domains of the antibody binds the NK cell (such as through an NK cell surface antigen) and one or more antigen binding domains of the antibody binds one or more target antigens. The multispecific antibody may be bispecific, trispecific, or tetraspecific, for example. In cases wherein the antibody is trispecific or tetraspecific, the additional antigen binding domains may bind other cells, such as stem cells.

In particular embodiments, the antibodies may bind any NK cell surface antigen (that may or may not be receptors) on NK cells, such as CD16 (including CD16a or CD16b), CD56, a c-type lectin such as NKG2D, NKG2C, a costimulatory molecule such as CS1, DNAM, 2B4, CD2, an NCR, or KIR, and redirect the NK cells to a target, thus increasing the response and specificity against different tumors.

Generation of the complexes may be by any suitable means, such that the conditions are sufficient for the appropriate region of the antibody to bind the appropriate surface region of the NK cell. Any particular medium may be utilized, in certain instances. In specific cases, Plasma-Lyte A and/or human serum albumin are utilized, wherein in other cases they are not. Once the complexes are formed in culture, they may or may not be washed prior to administration to the subject, such as through infusion. In alternative embodiments, the NK cells and the antibodies are administered separately, and the complexes form in vivo.

C. Pre-Activation

In some embodiments, the NK cells are pre-activated prior to administration to a recipient individual. The pre-activation step may or may not occur before any expansion step. In specific embodiments, the NK cells are pre-activated with one or more cytokines, and in specific embodiments, the NK cells are pre-activated with one or more of IL-12, IL-15, IL-2, and IL-18 and including two, three, or more. In cases wherein less than all three of IL-12, IL-15, IL-2, and IL-18 are utilized, it may be that IL-12 and IL-15 but not IL-18; or IL-12 and IL-18 but not IL-15; or IL-15 and IL-18 but not IL-12. IL-2 may or may not be substituted for IL-15.

In particular embodiments, the pre-activation cytokines may be IL-12, IL-15, and IL-18. One or more additional cytokines may be used for the pre-activation step. The pre-activation may be for a short period of time such as 5-72 hours, such as 10-50 hours, particularly 10-20 hours, such as 12, 13, 14, 15, 16, 17, 18, 19, or 20 hours, and specifically about 16 hours in some cases. The pre-activation culture may comprise IL-18 and/or IL-15 at a concentration of 10-100 ng/mL, such as 40-60 ng/mL, particular 45, 46, 47, 48, 49, 50, 51, 52, 53, 54, or 55 ng/ml, specifically about 50 ng/mL. In some cases, the pre-activation culture comprises IL-12 at a concentration of 0.1-150 ng/mL, including at a concentration of 1-20 ng/ml, such as a concentration of 10 ng/mL. In alternative embodiments the NK cells may be stimulated with IL-2, or other cytokines that bind the common gamma-chain (e.g., IL-7, IL-21, and others), and this may be in addition to IL-12, IL-15, and IL-18 or as an alternative to one or more of them. In such cases, the pre-activation culture may comprise IL-12 at a concentration of 0.1-150 ng/ml, such as 0.5-50 ng/mL, particularly 1-20 ng/ml, such as 2, 3, 4, 5, 6, 7, 8, 9, 10, 11, 12, 13, 14, or 15 ng/ml, specifically about 10 ng/mL.

D. Expansion

In particular embodiments, NK cells are expanded to increase their quantity prior to administration to an individual in need thereof. The expanded cells may or may not be derived from pre-activated NK cells such that a pre-activation step may occur before an expansion step. The NK cell expansion step may be of any suitable such that the NK cell population is expanded, but in specific cases the expansion step utilizes particular one or more reagents, such as in culture, to enhance their expansion. In certain cases the NK cells may not be expanded. IL-2 or IL-15 or IL-18 or any combination of the cytokines may be added to the expansion culture before or during expansion. The NK cells can be expanded ex vivo in flasks or in one of several different bioreactor configurations with continuous perfusion of media/additives, in specific embodiments.

In specific cases, the NK cells (whether pre-activated or not) may be washed (e.g., with PBS or Plasma Lyte or human serum albumin or culture media or combinations thereof) prior to and/or after expansion, such as 2, 3, 4, or 5 times, specifically 3 times. In particular embodiments, the NK cells are expanded in the presence of artificial antigen presenting cells (aAPCs). The aAPCs may be engineered to express CD137 ligand and/or a membrane-bound cytokine. The membrane-bound cytokine may be membrane-bound IL-21 (mIL-21) or membrane-bound IL-15 (mIL-15). In particular embodiments, the aAPCs are engineered to express CD137 ligand and mIL-21. The aAPCs may be derived from cancer cells, such as leukemia cells. The aAPCs may not express endogenous HLA class I, II, or CD1d molecules. They may express ICAM-1 (CD54) and LFA-3 (CD58) or CD48. In particular, the aAPCs may be K562 cells, such as K562 cells engineered to express CD137 ligand and mIL-21. The engineering may be by any method known in the art, such as retroviral transduction, although any viral or non-viral vector may be utilized. The aAPCs may or may not be irradiated. The expansion may be for a particular duration in time, such as for about 2-30 days, such as 3-20 days, particularly 12-16 days, such as 12, 13, 14, 15, 16, 17, 18, or 19 days, specifically about 14 days. The pre-activated NK cells and aAPCs may be present at a ratio of about 3:1-1:3, such as 2:1, 1:1, 1:2, specifically about 1:2. The expansion culture may further comprise one or more cytokines to promote expansion, such as IL-2. The IL-2 may be present at a concentration of about 10-500 U/mL, such as 100-300 U/mL, particularly about 200 U/mL. The IL-2 may be replenished in the expansion culture, including at a certain frequency, such as every 2-3 days. The aAPCs may be added to the culture at least a second time, such as at about 7 days of expansion. Any cytokine(s) used in the pre-activation and/or expansion steps may be recombinant human cytokines.

Following expansion, the NK cells may be immediately utilized in any manner, such as complexed with one or more antibodies, or they may be stored, such as by cryopreservation. In certain aspects, the cells may be propagated for days, weeks, or months ex vivo as a bulk population within about 1, 2, 3, 4, or 5 days.

Activated and/or expanded NK cells can secrete type I cytokines, such as interferon-y, tumor necrosis factor-a and granulocyte-macrophage colony-stimulating factor (GM-CSF), which activate both innate and adaptive immune cells as well as other cytokines and chemokines. The measurement of these cytokines can be used to determine the activation status of NK cells. In addition, other methods known in the art for determination of NK cell activation may be used for characterization of the NK cells of the present disclosure.

Thus, with respect to particular pre-activation and expansion aspects of the disclosure, in specific embodiments the NK cells pre-activated with IL-12, IL15, and IL-18 followed by expansion with aAPCs, such as K562 cells expressing mIL-21 and CD137 ligand, provide a highly potent cellular product. Thus, methods are provided using the present NK cells for the treatment of various diseases, such as immunotherapy of patients with cancer. In an exemplary method, the isolated NK cells may be subjected to a brief period, such as about 16 hours, of pre-activation with a combination of cytokines, such as interleukin-12 (IL-12), IL-15, and/or IL-18, followed by expansion using artificial antigen presenting cells (aAPCs), such as K562 feeder cells expressing membrane-bound IL-21 and CD137 ligand, and/or exogenous IL-2. IL-2 or IL-15 or IL-18 or any combination of the cytokines may be added to the expansion culture at least a second time.

E. Other Modifications

In some embodiments, the NK cells are modified compared to natural NK cells in one or more ways. The NK cells may be manipulated by the hand of man to be engineered to express one or more heterologous proteins. Alternatively, or in addition, the NK cells may be modified to have reduced or inhibited expression of one or more endogenous genes. In other cases, the NK cells is modified to express one or more cytokines or a suicide gene.

1. Engineered Antigen Receptors

The NK cells may be genetically modified to express one or more engineered antigen receptors, including at least one or more chimeric antigen receptors (CARs) and/or one or more TCRs. In specific embodiments, the engineered antigen receptors are directed to target one or more cancer antigens, and the antigen may or may not be the same antigen to which the multispecific antibody (e.g., engager) is directed.

The NK cells may be modified to encode at least one CAR, and the CAR may be first generation, second generation, or third or a subsequent generation, for example. The CAR may or may not be bispecific for two or more different antigens. The CAR may comprise one or more costimulatory domains. NK cells may also be modified to express a receptor to enhance their binding to the antibody, such as CD16, CD32 and/or CD64 receptor. Each costimulatory domain may comprise the costimulatory domain of any one or more of, for example, members of the TNFR superfamily, CD28, CD137 (4-1BB), CD134 (OX40), DAP10, DAP12, CD27, CD2, CD5, ICAM-1, LFA-1 (CD11a/CD18), Lck, TNFR-I, TNFR-II, Fas, CD30, CD27, NKG2D, 2B4M, CD40 or combinations thereof, for example. In specific embodiments, the CAR comprises CD3zeta. In certain embodiments, the CAR lacks one or more specific costimulatory domains; for example, the CAR may lack 4-1BB and/or lack CD28.

In particular embodiments, the CAR polypeptide in the cells comprises an extracellular spacer domain that links the antigen binding domain and the transmembrane domain, and this may be referred to as a hinge. Extracellular spacer domains may include, but are not limited to, Fc fragments of antibodies or fragments or derivatives thereof, hinge regions of antibodies or fragments or derivatives thereof, CH2 regions of antibodies, CH3 regions antibodies, artificial spacer sequences or combinations thereof. Examples of extracellular spacer domains include but are not limited to CD8-alpha hinge, CD28, artificial spacers made of polypeptides such as Gly3, or CH1, CH3 domains of IgGs (such as human IgG1 or IgG4). In specific cases, the extracellular spacer domain may comprise (i) a hinge, CH2 and CH3 regions of IgG4, (ii) a hinge region of IgG4, (iii) a hinge and CH2 of IgG4, (iv) a hinge region of CD8-alpha or CD4, (v) a hinge, CH2 and CH3 regions of IgG1, (vi) a hinge region of IgG1 or (vii) a hinge and CH2 of IgG1, (viii) a hinge region of CD28, or a combination thereof. In specific embodiments, the hinge is from IgG1 and in certain aspects the CAR polypeptide comprises a particular IgG1 hinge amino acid sequence or is encoded by a particular IgG1 hinge nucleic acid sequence.

The transmembrane domain in the CAR may be derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules, such as DAP10 or DAP12.

Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine may be found at each end of a synthetic transmembrane domain.

In certain embodiments, the CAR may be co-expressed with one or more cytokines to improve persistence when there is a low amount of tumor-associated antigen. For example, the CAR may be co-expressed with one or more cytokines, such as IL-7, IL-2, IL-15, IL-12, IL-23, IL-18, IL-21, IL-7, GMCSF, or a combination thereof. In some embodiments, the NK cells expressing the CAR are engineered to express one or more heterologous cytokines and/or are engineered to upregulate normal expression of one or more heterologous cytokines. The cells may or may not be transduced or transfected for one or more cytokines on the same vector as other genes.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells of any kind as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV. Non-viral vectors include plasmids, transposons, nanoparticles, liposome, lipids, metals, or a

In some embodiments, the genetically engineered antigen receptors include recombinant TCRs and/or TCRs cloned from naturally occurring T cells. A “T cell receptor” or “TCR” refers to a molecule that contains a variable a and β chains (also known as TCRα and TCRβ, respectively) or a variable y and 8 chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (sec, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the B-chain can contain a further hypervariability (HV4) region.

In some embodiments, the TCR chains contain a constant domain. For example, like immunoglobulins, the extracellular portion of TCR chains (e.g., a-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., a-chain constant domain or Cₐ, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the a and B chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). Sec, e.g., tumor antigens (scc, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

The CARs and TCRs of the disclosure target one or more particular antigens. Among the antigens targeted by the genetically engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B. T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may be targeted in the present method. The antigen may be associated with certain cancer cells but not associated with non-cancerous cells, in some cases. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include CD19, EBNA, CD123, HER2, CA-125, TRAIL/DR4, CD20, CD70, CD38, trop2, HLA-G, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, −10, GAGE-1,−2,−8, GAGE-3,−4,−5,-6,−7B, NA88-A, MC1R, mda-7, gp75, Gp100, PSA, PSM, Tyrosinase, tyrosinase-related protein, TRP-1. TRP-2, ART-4, CAMEL, CEA, Cyp-B, hTERT, hTRT, iCE, MUCI, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CDK-4/m, ELF2M, GnT-V. G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mber-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAGIB, SUNC1, and LRRN1. Examples of sequences for antigens are known in the art, for example, in the GenBank® database: CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRVIII (Accession No. NG_007726.3), MUCI (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. Sec, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

2. Suicide Gene

In particular embodiments, a suicide gene is utilized in conjunction with the antibody combination therapy to control its use and allow for termination of the cell therapy at a desired event and/or time. The suicide gene is employed in transduced cells for the purpose of eliciting death for the transduced cells when needed. The cells of the present disclosure that have been modified to harbor a vector encompassed by the disclosure may comprise one or more suicide genes. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product.

In some cases, the cell therapy may be subject to utilization of one or more suicide genes of any kind when an individual receiving the cell therapy and/or having received the cell therapy shows one or more symptoms of one or more adverse events, such as cytokine release syndrome, neurotoxicity, anaphylaxis/allergy, and/or on-target/off tumor toxicities (as examples) or is considered at risk for having the one or more symptoms, including imminently. The use of the suicide gene may be part of a planned protocol for a therapy or may be used only upon a recognized need for its use. In some cases the cell therapy is terminated by use of agent(s) that targets the suicide gene or a gene product therefrom because the therapy is no longer required.

Utilization of the suicide gene may be instigated upon onset of at least one adverse event for the individual, and that adverse event may be recognized by any means, including upon routine monitoring that may or may not be continuous from the beginning of the cell therapy. The adverse event(s) may be detected upon examination and/or testing. In cases wherein the individual has cytokine release syndrome (which may also be referred to as cytokine storm), the individual may have elevated inflammatory cytokine(s) (merely as examples: interferon-gamma, granulocyte macrophage colony-stimulating factor, IL-10, IL-6 and TNF-alpha); fever; fatigue; hypotension; hypoxia, tachycardia; nausea; capillary leak; cardiac/renal/hepatic dysfunction; or a combination thereof, for example. In cases wherein the individual has neurotoxicity, the individual may have confusion, delirium, aplasia, and/or seizures. In some cases, the individual is tested for a marker associated with onset and/or severity of cytokine release syndrome, such as C-reactive protein, IL-6, TNF-alpha, and/or ferritin

Examples of suicide genes include engineered nonsecretable (including membrane bound) tumor necrosis factor (TNF)-alpha mutant polypeptides (see PCT/US19/62009, which is incorporated by reference herein in its entirety), and they may be affected by delivery of an antibody that binds the TNF-alpha mutant. Examples of suicide gene/prodrug combinations that may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be utilized. Other suicide genes include CD20, CD52, inducible caspase 9, purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP), as examples.

In particular embodiments, vectors that encode the CAR, or any vector in a NK cell encompassed herein, include one or more suicide genes. The suicide gene may or may not be on the same vector as a CAR. In cases wherein the suicide gene is present on the same vector as the CAR, the suicide gene and the CAR may be separated by an IRES or 2A element, for example.

3. Cytokines

In some embodiments, the cells expressing the NK cells are engineered to express one or more heterologous cytokines and/or are engineered to upregulate normal expression of one or more heterologous cytokines. The cells may or may not be transduced or transfected for one or more cytokines on the same vector as other genes.

One or more cytokines may be co-expressed from a vector, including as a separate polypeptide from the engineered antigen receptor and/or suicide gene. Interleukin-15 (IL-15), for example, is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death (AICD). In addition to IL-15, other cytokines are envisioned. These include, but are not limited to, cytokines, chemokines, and other molecules that contribute to the activation and proliferation of cells used for human application. NK cells expressing IL-15 are capable of continued supportive cytokine signaling, which is useful for their survival post-infusion.

In specific embodiments, the cells express one or more exogenously provided cytokines. As one example, the cytokine is IL-15, IL-12, IL-2, IL-18, IL-21, IL-23, GMCSF, or a combination thereof. The cytokine may be exogenously provided to the NK cells because it is expressed from an expression vector within the cell. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as a suicide gene and/or as the CAR. In some embodiments, the present disclosure concerns co-utilization of a CAR with IL-15 and optionally a suicide gene.

4. Knockout or Knockdown of Engogenous Genes

The NK cell production process of the disclosure may include gene editing of the NK cells to remove 1, 2, 3, 4, 5, 6, 7, 8, 9, 10, or more endogenous genes in the NK cells. In some cases the gene editing occurs in NK cells expressing one or more heterologous antigen receptors, whereas in other cases the gene editing occurs in NK cells that do not express a heterologous antigen receptor but that ultimately will express one or more heterologous antigen receptors, in at least some cases. In particular embodiments, the NK cells that are gene edited are expanded NK cells.

In particular cases, one or more endogenous genes of the NK cells are modified, such as disrupted in expression where the expression is reduced in part or in full. In specific cases, one or more genes are knocked down or knocked out using processes of the disclosure. In specific cases, multiple genes are knocked down or knocked out in the same step as processes of the disclosure. The genes that are edited in the NK cells may be of any kind, but in specific embodiments the genes are genes whose gene products inhibit activity and/or proliferation of NK cells. In specific cases the genes that are edited in the NK cells allow the NK cells to work more effectively in a tumor microenvironment. In specific cases, the genes are one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL 10R. TDAG8, CD5, CD7, SLAMF7, CD38, LAG3, TCR, beta2-microglobulin, HLA, CD73, and CD39. In specific embodiments, the TGFBR2 gene is knocked out or knocked down in the NK cells.

In some embodiments, the gene editing is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. Methods of utilizing a CRISPR system are well known in the art

F. Cryopreservation

In particular cases, NK cells and/or antibodies of the disclosure are preserved in a cryopreservation medium composition comprising at least one cryoprotectant, a serum (human or animal serum) or a non-serum alternative to serum (not human serum or animal serum), and at least one cytokine and/or at least one growth factor. In some cases, the cryoprotectant is dimethyl sulfoxide (DMSO), glycerin, glycerol, hydroxyethol starch, or a combination thereof. The non-serum alternative may be of any kind, including at least platelet lysate and/or a blood product lysate (for example, human serum albumin). In embodiments of the composition wherein one or more (including two or more) cytokines are utilized, the cytokine may be a natural or a recombinant or a synthetic protein. At least one of the cytokines may be an Food and Drug Administration (FDA)-approved cytokine. Examples of cytokines and growth factors include at least IL-1, IL-2, IL-3, IL-4, IL-6, IL-7, IL-9, IL-10, IL-12, IL-13, IL-15, IL-17, IL-18, IL-21, IL-22, interferon, tumor necrosis factor, stem cell factor, FLT3-ligand, APRIL, thrombopoietin, erythropoietin, or a combination thereof. For serum embodiments, the serum may be an animal-derived serum, such as human serum (including human AB serum) or bovine serum. DMSO and other cryoprotectants, when utilized may comprise 4-10%, 4-6%, 4-8%, 5-10%, 5-8%, 6-10%, 6-8%, 8-10%, and so forth, of the composition. For embodiments wherein serum is employed, the serum may comprise 5-99%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-99%, 10-95%, 10-90%, 10-85%, 10-80%, 10-75%, 10-70%, 10-65%, 10-60%, 10-55%, 10-50%, 10-45%, 10-40%, 10-35%, 10-30%, 10-25%, 10-20%, 10-15%, 20-99%, 20-95%, 20-90%. 20-85%, 20-80%, 20-75%, 20-70%, 20-65%, 20-60%, 20-55%, 20-50%, 20-45%, 20-40%, 20-35%, 20-30%, 20-25%, 30-99%, 30-95%, 30-90%, 30-85%, 30-80%, 30-75%, 30-70%, 30-65%, 30-60%, 30-55%, 30-50%, 30-45%, 30-40%, 30-35%, 40-99%, 40-95%, 40-90%, 40-85%, 40-80%, 40-75%, 40-70%, 40-65%, 40-60%, 40-55%, 40-50%, 40-45%, 50-99%, 50-95%, 50-90%, 50-85%, 50-80%, 50-75%, 50-70%, 50-65%, 50-60%, 50-55%, 60-99%, 60-95%, 60-90%, 60-85%, 60-80%, 60-75%, 60-70%, 60-65%, 70-99%, 70-95%, 70-90%, 70-85%, 70-80%, 70-75%, 80-99%, 80-95%, 80-90%, 80-85%, 90-99%, 90-95%, or 95-99% of the composition. The composition may comprise at least or no more than 10, 15, 20, 25, 30, 35, 40, 45, 50, 55, 60, 65, 70, 75, 80, 85, 90, 91, 92, 93, 94, 95, 96, 97, 98, or 99% of serum. In specific embodiments, the composition comprises platelet lysate that may be at any concentration in the composition, but in certain embodiments the platelet lysate comprises 5-99%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-99%, 10-95%, 10-90%, 10-85%, 10-80%, 10-75%, 10-70%, 10-65%, 10-60%, 10-55%, 10-50%, 10-45%, 10-40%, 10-35%, 10-30%, 10-25%, 10-20%, 10-15%, 20-99%, 20-95%, 20-90%. 20-85%, 20-80%, 20-75%, 20-70%, 20-65%, 20-60%, 20-55%, 20-50%, 20-45%, 20-40%, 20-35%, 20-30%, 20-25%, 30-99%, 30-95%, 30-90%, 30-85%, 30-80%, 30-75%, 30-70%, 30-65%, 30-60%, 30-55%, 30-50%, 30-45%, 30-40%, 30-35%, 40-99%, 40-95%, 40-90%, 40-85%, 40-80%, 40-75%, 40-70%, 40-65%, 40-60%, 40-55%, 40-50%, 40-45%, 50-99%, 50-95%, 50-90%, 50-85%, 50-80%, 50-75%, 50-70%, 50-65%, 50-60%, 50-55%, 60-99%, 60-95%, 60-90%, 60-85%, 60-80%, 60-75%, 60-70%, 60-65%, 70-99%, 70-95%, 70-90%, 70-85%, 70-80%, 70-75%, 80-99%, 80-95%, 80-90%, 80-85%, 90-99%, 90-95%, or 95-99% of the composition. The composition may comprise at least or no more than 10, 15, 20, 25, 30, 35, 40, 45, 50, 55, 60, 65, 70, 75, 80, 85, 90, 91, 92, 93, 94, 95, 96, 97, 98, or 99% of platelet lysate.

The composition may have certain concentrations of components, including cytokines and/or growth factors. In specific cases, any cytokine, including IL-2, IL-21, and/or IL-15, for example, are present in the composition in a particular concentration. The IL-2 may be present at a concentration of 1-5000, 1-1000, 1-500, 1-100, 100-5000, 100-500, 500-5000, 500-1000, or 1000-5000 U/mL, for example. In a specific case, the IL-2 is present at a concentration in the composition of at least or no more than 100, 200, 300, 400, 500, 600, 700, 800, 900, or 1000 U/mL. In specific embodiments, IL-21 is present in the composition at a concentration of 10-3000, 10-2000, 10-1000, 10-500, 10-100, 100-3000, 100-2000, 100-1000, 500-3000, 500-2000, 500-1000, 1000-3000, 1000-2000, or 2000-3000 ng/mL. The IL-21 may be in a concentration in the composition of at least or nor more than 1, 5, 10, 20, 30, 40, 50, 60, 70, 80, 90, 100, 150, 200, 250, 300, 350, 400, 450, 500, 750, 1000, 1250, 1500, 1750, 2000, 2250, 2500, 2750, or 3000 ng/mL. IL-15 may be present in the composition at a concentration of 1-2000, 1-1000, 1-500, 1-100, 100-2000, 100-1000, 100-500, 500-2000, 500-1000, or 1000-2000 ng/mL. IL-15 may be present in the composition at a concentration of at least or no more than 10, 50, 100, 500, 1000, 1500, or 2000 ng/mL.

Compositions as encompassed herein that comprise at least one cryoprotectant, a serum or a non-serum alternative to serum, and at least one cytokine and/or at least one growth factor may further comprise a plurality of immune cells and/or stem cells, each of any kind. In specific embodiments, the cells are NK cells, T cells, B cells, NKT cells derived from mature bone marrow or peripheral blood cells, cell lines such as tumor cell lines (e.g., NK92 or other NK lines), hematopoietic stem cells, induced pluripotent stem cells, MSCs (a population of cells alternatively called “mesenchymal stem cells” and “mesenchymal stromal cells” in the literature), or a mixture thereof, which can be derived from bone marrow, peripheral blood, skin, adipose tissue, or a combination thereof. In embodiments wherein NK cells are utilized, the NK cells may or may not be expanded NK cells. Embodiments of the disclosure also encompass pharmaceutical compositions that comprise any composition of the disclosure and a suitable pharmaceutically acceptable carrier.

In specific embodiments, the cells of the disclosure at any step are preserved in the following particular formulations for cryopreservation media:

50% RPMI; 25% dextran; 20% human albumin, 5% DMSO
50% RPMI; 25% dextran; 20% human albumin, 5% DMSO + IL-2/IL-15
35% RPMI; 40% dextran; 20% human albumin, 5% DMSO
35% RPMI; 40% dextran; 20% human albumin, 5% DMSO + IL-2/IL-15
32.5% RPMI; 40% dextran; 20% human albumin, 7.5% DMSO
32.5% RPMI; 40% dextran; 20% human albumin, 7.5% DMSO + IL-2/IL-15
50% PlasmaLyte; 25% dextran; 20% human albumin, 5% DMSO
50% PlasmaLyte; 25% dextran; 20% human albumin, 5% DMSO + IL-2/IL-15
35% PlasmaLyte; 40% dextran; 20% human albumin, 5% DMSO
35% PlasmaLyte; 40% dextran; 20% human albumin, 5% DMSO + IL-2/IL-15
32.5% PlasmaLyte; 40% dextran; 20% human albumin, 7.5% DMSO
32.5% PlasmaLyte; 40% dextran; 20% human albumin, 7.5% DMSO + IL-2/IL-15
70% PlasmaLyte; 25% dextran; 5% DMSO + IL-2/IL-15
90% PLT Lys + 10% DMSO + IL-2/IL-15
50% PLT lys + 25% dextran + 20% human albumin + 5% DMSO + IL-2/IL-15
50% AB serum + 25% dextran + 20% human albumin + 5% DMSO + IL-2/IL-15
50% PLT lys + 25% dextran + 20% human albumin + 5% DMSO + IL-2/IL-21
50% RPMI + 25% dextran + 20% human albumin + 5% DMSO + IL-2/IL-21
50% PlasmaLyte + 25% dextran + 20% human albumin + 5% DMSO + IL-2/IL-21
50% AB serum + 25% dextran + 20% human albumin + 5% DMSO + IL-2/IL-21
50% PLT Lys; 25% Dextran in NACL; 20% human albumin; 5% DMSO + IL-2/IL-15
50% PLT Lys; 25% Dextran in Dextrose; 20% human albumin; 5% DMSO + IL-2/IL-15
25% PLT Lys; 50% Dextran in NACL; 20% human albumin; 5% DMSO + IL-2/IL-15
25% PLT Lys; 50% Dextran in Dextrose; 20% human albumin; 5% DMSO + IL-2/IL-15
25% Dextran in NACL; 70% human albumin; 5% DMSO + IL-2/IL-15
25% Dextran in Dextrose; 70% human albumin; 5% DMSO + IL-2/IL-15
50% Dextran in NACL; 45% human albumin; 5% DMSO + IL-2/IL-15
50% Dextran in Dextrose; 45% human albumin; 5% DMSO + IL-2/IL-15
50% Plasmalyte; 45% human albumin; 5% DMSO + IL-2/IL-15
25% Plasmalyte; 70% human albumin; 5% DMSO + IL-2/IL-15
Crystor Cell preservation Media CS10 (1% to 100%)
Crystor Cell preservation Media CS10 (1% to 100%) + IL-2/15

Examples of particular formulations with certain concentrations may be utilized as follows:

50% Platelet lysate; 25% Dextran in NaCL; 20% human albumin; 5% DMSO; plus 10-
200 iu of interleukin 2 and/or 1-10 ng/ml of interleukin 15
50% Platelet lysate; 25% Dextran in Dextrose; 20% human albumin; 5% DMSO; plus 10-
200 iu of interleukin 2 and/or 1-10 ng/ml of interleukin 15
25% Platelet lysate; 50% Dextran in NaCL; 20% human albumin; 5% DMSO; plus 10-
200 iu of interleukin 2 and/or 1-10 ng/ml of interleukin 15
25% Platelet lysate; 50% Dextran in Dextrose; 20% human albumin; 5% DMSO; plus 10-
200 iu of interleukin 2 and/or 1-10 ng/ml of interleukin 15
25% Dextran in NaCL; 70% human albumin; 5% DMSO; plus 10-200 iu of interleukin 2
and/or 1-10 ng/ml of interleukin 15
25% Dextran in Dextrose; 70% human albumin; 5% DMSO; plus 10-200 iu of interleukin 2
and/or 1-10 ng/ml of interleukin 15
50% Dextran in NaCL; 45% human albumin; 5% DMSO; plus 10-200 iu of interleukin 2
and/or 1-10 ng/ml of interleukin 15
50% Dextran in Dextrose; 45% human albumin; 5% DMSO; plus 10-200 iu of interleukin 2
and/or 1-10 ng/ml of interleukin 15
50% Plasmalyte; 45% human albumin; 5% DMSO; plus 10-200 iu of interleukin 2 and/or 1-
10 ng/ml of interleukin 15
25% Plasmalyte; 70% human albumin; 5% DMSO; plus 10-200 iu of interleukin 2 and/or 1-
10 ng/ml of interleukin 15
90% Platelet lysate, 10% DMSO
90% Platelet lysate, 10% DMSO plus 10-200 iu of interleukin 2 and/or 1-10 ng/ml of
interleukin 15
Crystor Cell preservation Media CS10 (1% to 100%)
Crystor Cell preservation Media CS10 (1% to 100%) + IL-2

II. Methods of Use

Embodiments of the present disclosure concern methods for the use of the compositions comprising NK cells and antibodies provided herein for treating or preventing a medical disease or disorder. The method includes administering to the subject a therapeutically effective amount of the loaded, optionally pre-activated, and optionally expanded NK cells with the antibodies, thereby treating or preventing the disease in the subject, including reducing the risk of, reducing the severity of, and/or delaying the onset of the disease. In certain embodiments of the present disclosure, cancer or infection is treated by transfer of a composition comprising the NK cell population and antibodies. In at least some cases, because of their release of pro-inflammatory cytokines, NK cells may reverse the anti-inflammatory tumor microenvironment and increase adaptive immune responses by promoting differentiation, activation, and/or recruitment of accessory immune cell to sites of malignancy.

Cancers for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; cosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Particular embodiments concern methods of treatment of leukemia. Leukemia is a cancer of the blood or bone marrow and is characterized by an abnormal proliferation (production by multiplication) of blood cells, usually white blood cells (leukocytes). It is part of the broad group of diseases called hematological neoplasms. Leukemia is a broad term covering a spectrum of diseases. Leukemia is clinically and pathologically split into its acute and chronic forms.

Acute leukemia is characterized by the rapid proliferation of immature blood cells. This crowding makes the bone marrow unable to produce healthy blood cells. Acute forms of leukemia can occur in children and young adults. In fact, it is a more common cause of death for children in the U.S. than any other type of malignant disease. Immediate treatment is required in acute leukemia due to the rapid progression and accumulation of the malignant cells, which then spill over into the bloodstream and spread to other organs of the body. Central nervous system (CNS) involvement is uncommon, although the disease can occasionally cause cranial nerve palsies. Chronic leukemia is distinguished by the excessive build up of relatively mature, but still abnormal, blood cells. Typically taking months to years to progress, the cells are produced at a much higher rate than normal cells, resulting in many abnormal white blood cells in the blood. Chronic leukemia mostly occurs in older people, but can theoretically occur in any age group. Whereas acute leukemia must be treated immediately, chronic forms are sometimes monitored for some time before treatment to ensure maximum effectiveness of therapy.

Furthermore, the diseases are classified into lymphocytic or lymphoblastic, which indicate that the cancerous change took place in a type of marrow cell that normally goes on to form lymphocytes, and myelogenous or myeloid, which indicate that the cancerous change took place in a type of marrow cell that normally goes on to form red cells, some types of white cells, and platelets (see lymphoid cells vs. myeloid cells).

Acute lymphocytic leukemia (also known as acute lymphoblastic leukemia, or ALL) is the most common type of leukemia in young children. This disease also affects adults, especially those aged 65 and older. Chronic lymphocytic leukemia (CLL) most often affects adults over the age of 55. It sometimes occurs in younger adults, but it almost never affects children. Acute myelogenous leukemia (also known as acute myeloid leukemia, or AML) occurs more commonly in adults than in children. This type of leukemia was previously called “acute nonlymphocytic leukemia.” Chronic myelogenous leukemia (CML) occurs mainly in adults. A very small number of children also develop this disease.

Lymphoma is a type of cancer that originates in lymphocytes (a type of white blood cell in the vertebrate immune system). There are many types of lymphoma. According to the U.S. National Institutes of Health, lymphomas account for about five percent of all cases of cancer in the United States, and Hodgkin's lymphoma in particular accounts for less than one percent of all cases of cancer in the United States. Because the lymphatic system is part of the body's immune system, patients with a weakened immune system, such as from HIV infection or from certain drugs or medication, also have a higher incidence of lymphoma.

In certain embodiments of the disclosure, compositions comprising the NK cells and antibodies are delivered to an individual in need thereof, such as an individual that has cancer or an infection. In at least some cases, the cells can enhance the individual's immune system to attack the respective cancer or pathogenic cells. In some cases, the individual is provided with one or more doses of the compositions comprising the NK cells and antibodies. In cases where the individual is provided with two or more doses of the NK cells/antibodies, the duration between the administrations should be sufficient to allow time for propagation in the individual, and in specific embodiments the duration between doses is 1, 2, 3, 4, 5, 6, 7, or more days.

The source of NK cells that are pre-activated (optionally) and expanded may be of any kind, but in specific embodiments the cells are obtained from a bank of umbilical cord blood, peripheral blood, human embryonic stem cells, or induced pluripotent stem cells, for example. Suitable doses for a therapeutic effect would be at least 10⁵ or between about 10⁵ and about 10¹² cells per dose, for example, preferably in a series of dosing cycles. An exemplary dosing regimen consists of four one-weck dosing cycles of escalating doses, starting at least at about 10⁵ cells on Day 0, for example increasing incrementally up to a target dose of about 10¹² cells within several weeks of initiating an intra-patient dose escalation scheme. Suitable modes of administration include intravenous, subcutaneous, intracavitary (for example by reservoir-access device), intraperitoneal, and direct injection into a tumor mass.

The compositions comprising the NK cells and antibodies generated according to the present methods have many potential uses, including experimental and therapeutic uses. In particular, it is envisaged that such cell populations are useful in suppressing undesirable or inappropriate immune responses. In such methods, a small number of NK cells are removed from a patient and then manipulated and expanded ex vivo before reinfusing them into the patient. Examples of diseases which may be treated in this way are autoimmune diseases and conditions in which suppressed immune activity is desirable, e.g., for allo-transplantation tolerance. A therapeutic method could comprise obtaining NK cells from a mammal; expanding the NK cells ex vivo in accordance with the methods of the present methods as described herein; exposing under sufficient conditions the NK cells to the antibodies; and administering the compositions comprising the expanded NK cells/antibodies to the mammal to be treated.

A pharmaceutical composition of the present disclosure can be used alone or in combination with other well-established agents useful for treating cancer. Whether delivered alone or in combination with other agents, the pharmaceutical composition of the present disclosure can be delivered via various routes and to various sites in a mammalian, particularly human, body to achieve a particular effect. One skilled in the art will recognize that, although more than one route can be used for administration, a particular route can provide a more immediate and more effective reaction than another route. For example, intradermal delivery may be advantageously used over inhalation for the treatment of melanoma. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration.

In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus crythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and expanded NK cells/antibodies of the disclosure are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of NK cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. NK cells, such as immunosuppressive CD19+ cells, can be administered along with the antibodies prior to transplantation, concurrently with transplantation, and/or following transplantation. In some embodiments, the compositions comprising the NK cells and antibodies are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of the compositions comprising the NK cells and antibodies occurs 3-5 days prior to transplantation.

NK cells/antibodies administered to a patient that is receiving a transplant can be sensitized with antigens specific to the transplanted material prior to administration. According to this embodiment, the transplant recipient will have a decreased immune/inflammatory response to the transplanted material and, as such, the likelihood of rejection of the transplanted tissue is minimized. Similarly, with regard to the treatment of graft versus host disease, the NK cells can be sensitized with antigens specific to the host. According to this embodiment, the recipient will have a decreased immune/inflammatory response to self-antigens.

In a further embodiment, administration of a therapeutically effective amount of compositions comprising NK cells and antibodies to a subject treats or inhibits inflammation in the subject. Thus, the method includes administering a therapeutically effective amount of compositions comprising NK cells and antibodies to the subject to inhibit the inflammatory process. Examples of inflammatory disorders include, but are not limited to, asthma, encephalitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, and chronic inflammation resulting from chronic viral or bacterial infections. The methods disclosed herein can also be used to treat allergic disorders.

Administration of compositions comprising NK cells and antibodies can be utilized whenever immunosuppression or inhibition of inflammation is desired, for example, at the first sign or symptoms of a disease or inflammation. These may be general, such as pain, edema, elevated temperature, or may be specific signs or symptoms related to dysfunction of affected organ(s). For example, in renal transplant rejection there may be an elevated serum creatinine level, whereas in GVHD, there may be a rash, and in asthma, there may be shortness of breath and wheezing.

Administration of compositions comprising NK cells and antibodies can also be utilized to prevent immune-mediated disease in a subject of interest. For example, compositions comprising NK cells and antibodies can be administered to a subject that will be a transplant recipient prior to the transplantation. In another example, compositions comprising NK cells and antibodies are administered to a subject receiving allogeneic bone marrow transplants without T cell depletion. In a further example, compositions comprising NK cells and antibodies can be administered to a subject with a family history of diabetes. In other example, compositions comprising NK cells and antibodies are administered to a subject with asthma in order to prevent an asthma attack. In some embodiments, a therapeutically effective amount of compositions comprising NK cells and antibodies is administered to the subject in advance of a symptom. The administration of the compositions comprising NK cells and antibodies results in decreased incidence or severity of subsequent immunological event or symptom (such as an asthma attack), or improved patient survival, compared to patients who received other therapy not including compositions comprising NK cells and antibodies.

In certain embodiments, the compositions comprising NK cells and antibodies are administered in combination with a second therapeutic agent. For example, the second therapeutic agent may comprise T cells, an immunomodulatory agent, a monoclonal antibody, a chemotherapeutic agent, hormone(s), drugs of any kind, surgery, radiation, etc. In non-limiting examples, the immunomodulatory agent is lenalidomide, the monoclonal antibody is rituximab, ofatumumab, or lumiliximab, and the chemotherapeutic agent is fludarabine or cyclophosphamide.

A composition of the present disclosure can be provided in unit dosage form wherein each dosage unit, e.g., an injection, contains a predetermined amount of the composition, alone or in appropriate combination with other active agents. The term unit dosage form as used herein refers to physically discrete units suitable as unitary dosages for human and animal subjects, each unit containing a predetermined quantity of the composition of the present disclosure, alone or in combination with other active agents, calculated in an amount sufficient to produce the desired effect, in association with a pharmaceutically acceptable diluent, carrier, or vehicle, where appropriate. The specifications for the unit dosage forms of the present disclosure depend on the particular pharmacodynamics associated with the pharmaceutical composition in the particular subject.

Desirably an effective amount or sufficient number of the isolated transduced NK cells is present in the composition and introduced into the subject such that long-term, specific, anti-tumor responses are established to reduce the size of a tumor or eliminate tumor growth or regrowth than would otherwise result in the absence of such treatment. Desirably, the amount of NK cells reintroduced into the subject causes a 10%, 20%, 30%, 40%, 50%, 60%, 70%, 80%, 90%, 95%, 98%, or 100% decrease in tumor size when compared to otherwise same conditions wherein the NK cells are not present.

Accordingly, the amount of compositions comprising NK cells and antibodies administered should take into account the route of administration and should be such that a sufficient number of the compositions comprising NK cells and antibodies will be introduced so as to achieve the desired therapeutic response. Furthermore, the amounts of each active agent included in the compositions described herein (e.g., the amount per each cell to be contacted or the amount per certain body weight) can vary in different applications. In general, the concentration of NK cells desirably should be sufficient to provide in the subject being treated at least from about 1×10⁶ to about 1×10¹² NK cells, even more desirably, from about 1×10⁷ to about 5×10¹⁰ NK cells, although any suitable amount can be utilized either above, e.g., greater than 5×10⁸ cells, or below, e.g., less than 1×10⁷ cells. The dosing schedule can be based on well-established cell-based therapies (see, e.g., U.S. Pat. No. 4,690,915), or an alternate continuous infusion strategy can be employed.

These values provide general guidance of the range of compositions comprising NK cells and antibodies to be utilized by the practitioner upon optimizing the method of the present disclosure for practice of the present methods. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on interindividual differences in pharmacokinetics, drug disposition, and metabolism. One skilled in the art readily can make any necessary adjustments in accordance with the exigencies of the particular situation.

III. Antibodies

Aspects of the disclosure relate to use of antibodies or functional fragments thereof in a composition also comprising certain NK cells. The term “antibody” refers to an intact immunoglobulin of any isotype, or a fragment thereof that can compete with the intact antibody for specific binding to the target antigen, such as through the antigen binding domain, and includes chimeric, humanized, fully human, monospecific, and multispecific (including at least bispecific and trispecific, and more) antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity. In specific embodiments, the antibody comprises an scFv.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, sec, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986 Sec, e.g., Epitope Mapping Protocols, supra. Additionally, antigenic regions of proteins can also be predicted and identified using standard antigenicity and hydropathy plots.

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al. Front Immunol. 2013; 4: 302; 2013)

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins, such as the following:

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as H1, H2 and H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, Aug. 1970); Chothia (as described in C. Chothia et al., “Conformations of immunoglobulin hypervariable regions,” Nature, vol. 342, no. 6252, pp. 877-883, Dec. 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, Jan. 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope; 2) Hydrogen-deuterium exchange and mass spectroscopy; 3) Polypeptide fragmentation and peptide mapping approaches in which one generates multiple overlapping peptide fragments from the full length of the polypeptide and evaluates the binding affinity of these peptides for the epitope; 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

Functional antibody fragments and antigen-binding fragments may be utilized. Certain aspects relate to antibody fragments, such as antibody fragments that bind to and/or neutralize inflammatory mediators. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CHI) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CHI domains; (ii) the Fd fragment type constituted with the VH and CHI domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, N Y (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015). The citations in this paragraph are all incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the VL, VH, CL and CHI domains. The term Fab′ fragment means a monovalent antigen-binding fragment of a monoclonal antibody that is larger than a Fab fragment. For example, a Fab′ fragment includes the VL, VH, CL and CHI domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CHI domains.

The term Fd fragment means a fragment of the heavy chain of a monoclonal antibody, which includes all or part of the VH, including the CDRs. An Fd fragment can further include CHI region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

In some cases, fragment crystallizable regions, Fc, are utilized. An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included. In some embodiments, antibodies that incorporate cytokines (including those referred to herein) may be utilized (such as with TRIKEs).

IV. Pharmaceutical Compositions

Pharmaceutical compositions of the present disclosure comprise an effective amount of compositions comprising NK cells and antibodies dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises the compositions will be known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein. “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The pharmaceutical compositions may comprise different types of carriers depending on whether it is to be administered in solid, liquid or aerosol form, and whether it need to be sterile for such routes of administration as injection. The presently disclosed compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (sec, for example, Remington's Pharmaceutical Sciences, 18th Ed. Mack Printing Company, 1990, incorporated herein by reference).

The compositions comprising the NK cells and antibodies may be formulated into a composition in a free base, neutral or salt form. Pharmaceutically acceptable salts, where appropriate include the acid addition salts, e.g., those formed with the free amino groups of a proteinaccous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the compositions of the present disclosure suitable for administration are provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include compositions comprising the NK cells and antibodies and optionally an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a nanoparticle or in a lipid vehicle. For example, the compositions comprising the NK cells and antibodies may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The therapeutic compositions comprising the NK cells and antibodies of the disclosure may be administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice, in certain embodiments, it is contemplated that doses in the range from 10 mg/kg to 200 mg/kg can affect the protective capability of these agents. Thus, it is contemplated that doses include doses of about 0.1, 0.5. 1. 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67. 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

A. Alimentary Compositions and Formulations

In particular embodiments of the present disclosure, the compositions comprising the NK cells and antibodies are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. Sec, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, compositions may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intravenously, intrathecally, intraventricularly, intra-tumorally, subcutaneously, or intraperitoneally U.S. Pat. Nos. 6,613,308; 5,466,468; 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other particular embodiments of the disclosure, the active compound compositions comprising the NK cells and antibodies may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

V. Combination Therapies

In certain embodiments, the compositions and methods of the present embodiments involve a cancer therapy that is additional to the compositions comprising the NK cells and antibodies. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, hormone therapy, or a combination of the foregoing. The additional therapy may be in the form of adjuvant or neoadjuvant therapy.

In some embodiments, the additional therapy is the administration of small molecule enzymatic inhibitor(s) or anti-metastatic agent(s). In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent(s). The additional therapy may be one or more of the chemotherapeutic agents known in the art.

An immune cell therapy (in addition to the compositions of the disclosure) may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from the composition(s) of the disclosure, one would generally ensure that a significant period of time did not expire between the time of each delivery, such that the two compounds would still be able to exert an advantageously combined effect on the patient. In such instances, it is contemplated that one may provide a patient with the immunotherapy therapy and the disclosed compositions within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trictylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurca; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

B. Radiotherapy

Other factors that cause DNA damage and have been used extensively include what are commonly known as y-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapy

The skilled artisan will understand that additional immunotherapies (outside of the disclosed cell therapy) may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells other than those having knockdown or knockout of TGF-beta R2.

Antibody-drug conjugates have emerged as a breakthrough approach to the development of cancer therapeutics. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons of any kind, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

VI. Kits of the Disclosure

In some embodiments, a kit that can include, for example, NK cells, optionally one or more media and components for the production of NK cells, one or more antibodies or reagent(s) to produce the antibodies, and so forth are provided. In some embodiments, formulations may comprise a cocktail of factors, including in a form suitable for combining with NK cells. The reagent system or any kit component may be packaged either in aqueous media or in lyophilized form, where appropriate. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The components of the kit may be provided as dried powder(s). When reagents and/or components are provided as a dry powder, the powder can be reconstituted by the addition of a suitable solvent. It is envisioned that the solvent may also be provided in another container means. The kits also will typically include a means for containing the kit component(s) in close confinement for commercial sale. Such containers may include injection or blow molded plastic containers into which the desired vials are retained. The kit can also include instructions for use, such as in printed or electronic format, such as digital format.

In specific embodiments, the kit may comprise one or more cytokines, including at least IL-12, IL-15, IL18, and/or IL-2, including in particular concentrations as described elsewhere herein. The kit may comprise any type of media, any component of a cryopreservation media, as described elsewhere herein. The kit may comprise cord blood (including pooled cord blood), antigen presenting cells of any kind, beads for depletion of particular NK cells (as described herein), vectors encoding one or more proteins as described herein. NK cells, antibodies or reagents to generate antibodies, etc.

Individual components may also be provided in a kit in concentrated amounts; in some embodiments, a component is provided individually in the same concentration as it would be in a solution with other components. Concentrations of components may be provided as 1x, 2x, 5x, 10x, or 20x or more.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Preparation and Use of Compositions

Compositions comprising particular NK cells and also antibodies that are able to bind the NK cells are prepared and, in particular cases, are given in an effective amount to an individual in need of treatment. In particular embodiments, NK cells that are autologous or allogeneic with respect to the individual are administered in an effective amount to the individual, and the NK cells are particularly cord blood cells. The NK cells may or may not be sourced from a cryopreservation repository and in some cases were cryopreserved in a particular cryopreservation medium. The NK cells are optionally pre-activated and optionally expanded prior to exposing an effective amount of the NK cells to the antibodies under conditions that allow the antibodies to bind the NK cells through the appropriate antigen. Following this, an effective amount of the compositions are provided to the individual in need of them.

### Example 2

Monoclonal Antibodies and Nk Cells

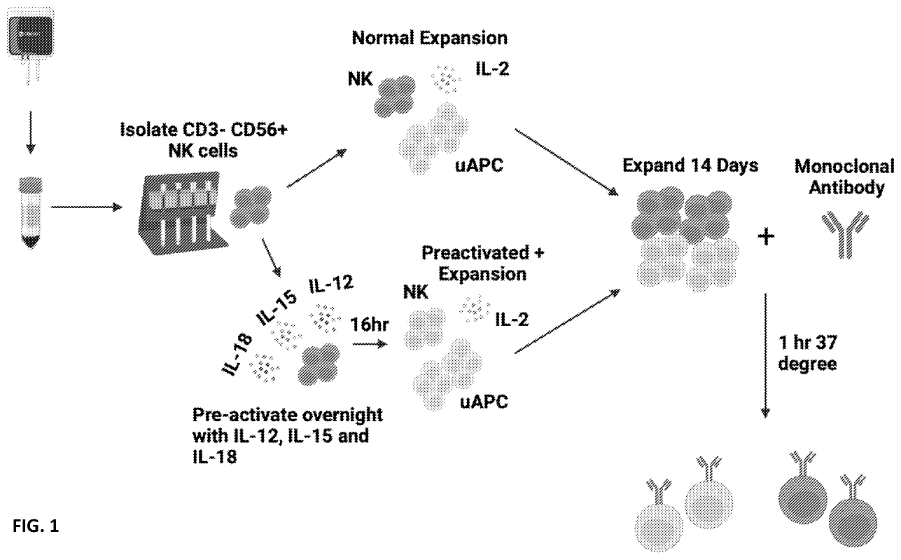

The present example encompasses preparation and use of NK cells preloaded with antibodies. FIG. 1 shows one example of an experimental procedure for NK cell expansion and antibody loading. NK cells may be obtained from blood that has been processed, and in specific embodiments certain NK cells are isolated, such as those that are CD3-CD56+, in specific embodiments. Optionally the NK cells are expanded, for example in the presence of IL-2 and universal antigen presenting cells or other feeder cells, and the expansion step may be of any suitable duration, such as up to about 14 days, in specific cases. In another optional embodiment, the NK cells are pre-activated prior to expansion. Although any pre-activation step may be utilized, in specific embodiments the pre-activation step encompasses exposure to one or more cytokines, such as one or more of IL-12, IL-15, and/or IL-18. The pre-activation step may take any suitable length of duration, but in specific embodiments it is up to about 16 hours. Following this step, the NK cells optionally may be expanded, including in the presence of universal antigen presenting cells and IL-2, for example. The cultured cell may be activated again with IL2, IL-18 or IL-15 or any combination for these cytokines during expansion. Following expansion, the NK cells are exposed to a suitable amount of antibody under sufficient conditions such that the antibody becomes bound to the outside of the NK cells though a particular antigen to which the antibody is directed. In specific cases, the antibody is a monoclonal antibody. The conditions for loading of the antibodies onto the NK cells may be under particular temperatures for a specific duration, such as one hour at 37° C.

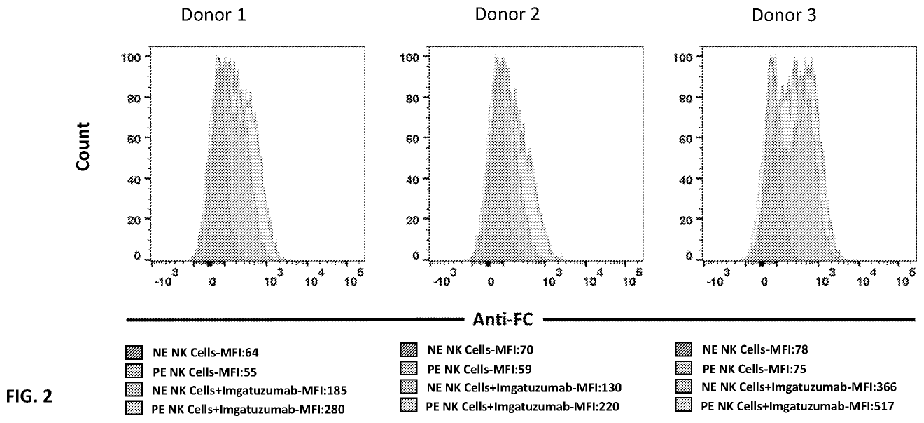

FIG. 2 provides validation of Margetuximab (anti-HER2 monoclonal antibody) binding to NK cells. NK cells were derived from cord blood and were either normally expanded (NE) with irradiated (100 Gy) uAPC feeder cells (2:1 feeder cell:NK ratio) and recombinant human IL-2 (200 U/ml) in 50:50 Click's/RPMI media or pre-activated with IL-12 (10 ng/ml), IL-15 (50 ng/ml), and IL-18 (50 ng/ml) for 16 hrs and then washed and expanded (PE) with irradiated uAPC and IL-2 (200 U/ml). NK cells were loaded with Margetuximab 1 μg/ml for one hour at 37ºC in Click's/RPMI media and were washed before validating the binding of Margetuximab to NK cells with flow cytometry. Non-loaded NE and PE NK cells were used as negative control. Margetuximab binding was detected by flow cytometry staining using Alexa-Fluor647 affinity-purified F(ab′)2fragment goat anti-human IgG (H+L) antibody. The graphs show that PE NK cells show higher level of Margetuximab binding compared to NE NK cells. Histograms provide data from 3 different cord blood NK cell donors.

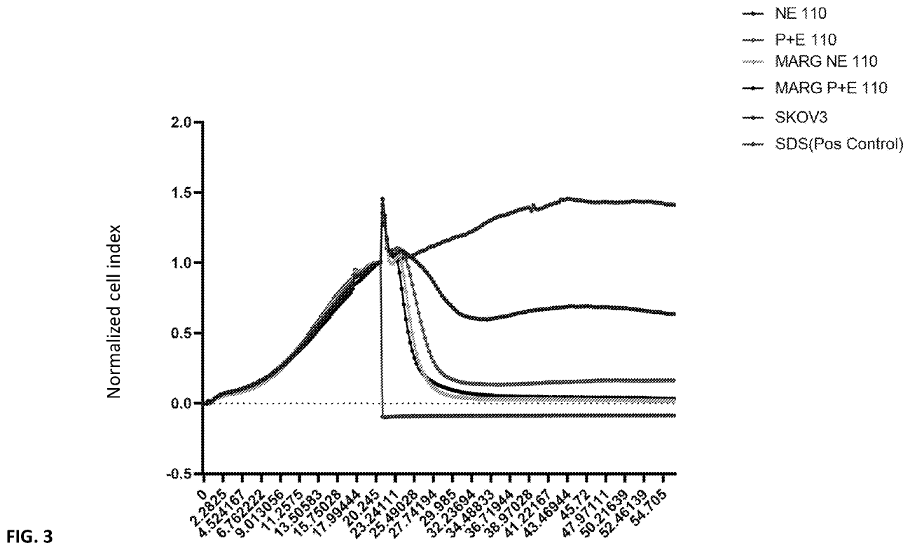

Margetuximab-loaded NK cells show enhanced cytotoxicity against HER2+tumor cells (FIG. 3). Provided therein is an Xcelligence cytotoxicity assay showing normalized cell index of HER2+SKOV3 (ovarian cancer) cultured alone, or with NK cells at 2:1 effector to target (E:T) ratio. NK cells were either normally expanded (NE) or preactivated and expanded (P+E). NE NK cells were cultured with irradiated (100 Gy) uAPC feeder cells (2:1 feeder cell:NK ratio) and recombinant human IL-2 (200 U/ml) in 50:50 Click's/RPMI media and PE cells were preactivated with IL-12 (10 ng/ml), IL-15 (50 ng/ml), and IL-18 (50 ng/ml) for 16 hrs and then washed and expanded with irradiated uAPC and IL-2 (200 U/ml). NK cells were either left unloaded or loaded with Margetuximab 1 μg/ml for one hour at 37° C. in Click's/RPMI media and then washed before the assay. Data indicate that Margetuximab loading enhances the cytotoxicity of NE and PE NK cells against tumor cells.

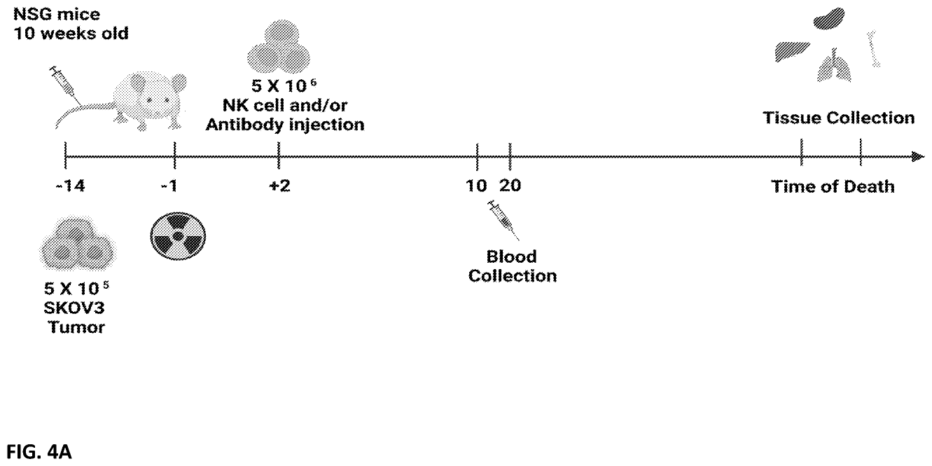

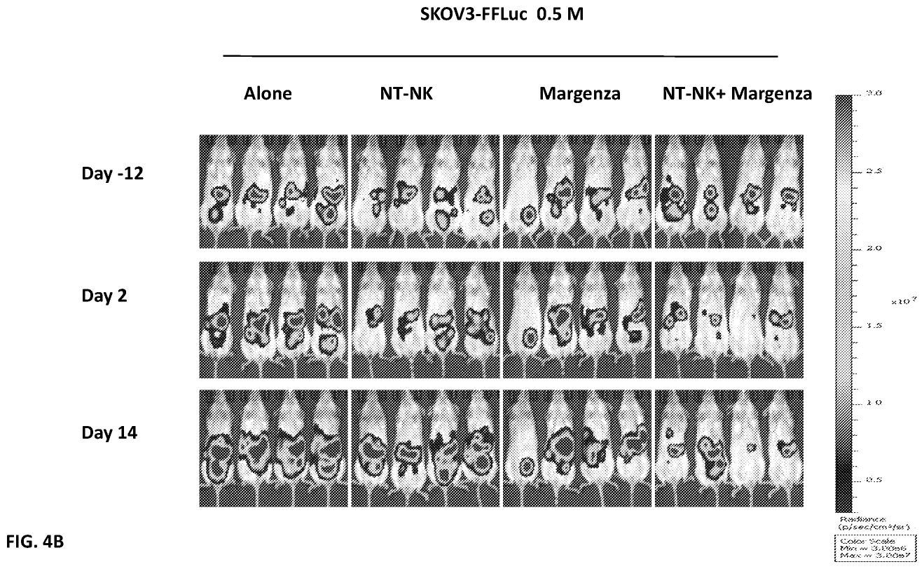

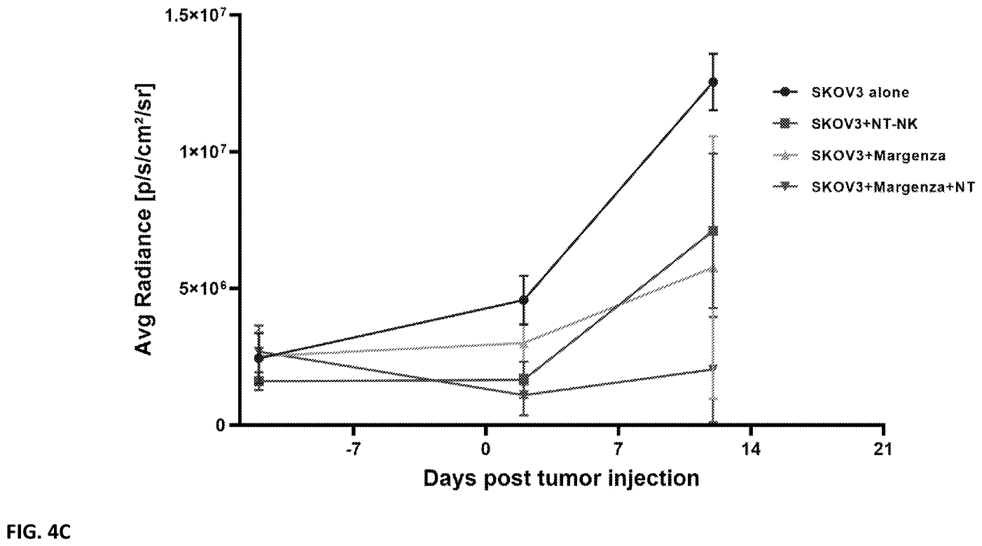

Margetuximab-loaded NK cells lead to enhanced tumor control in an NSG mouse model of HER2+ovarian cancer (SKOV3) (FIG. 4). In FIG. 4A, there is one example of a schema of experimental plan for SKOV3 mouse model. In FIG. 4B, bioluminescent imaging shows tumor growth over time in mice engrafted with SKOV3 transduced with Fireflyluciferase (FFluc) and either left untreated or treated with NK cells alone, Margetuximab alone, or NK cells pre-loaded with Margetuximab. FIG. 4C provides a graph showing average radiance over time of the four groups of mice described in panel 4A.

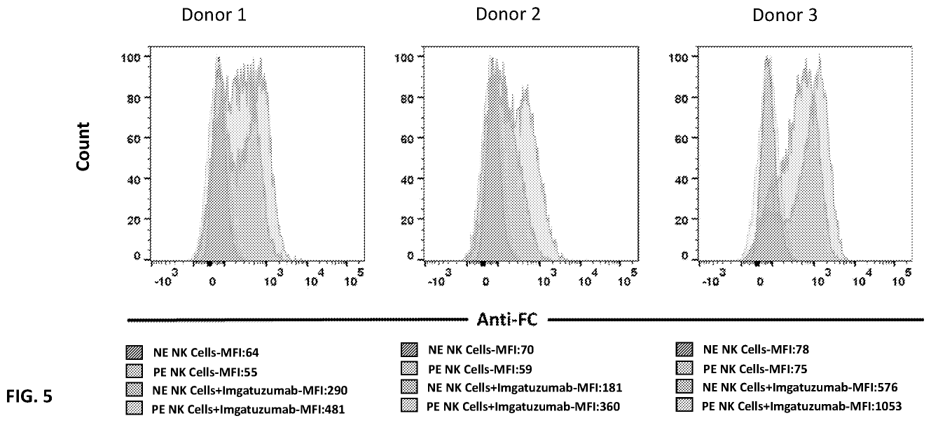

In FIG. 5, validation of Amivantamab (EGFR-MET bispecific antibody) binding to the NK cells is demonstrated. NK cells were derived from cord blood and were either normally expanded (NE) with irradiated (100 Gy) uAPC feeder cells (2:1 feeder cell:NK ratio) and recombinant human IL-2 (200 U/ml) in 50:50 Click's/RPMI media or pre-activated with IL-12 (10 ng/ml), IL-15 (50 ng/ml), and IL-18 (50 ng/ml) for 16 hrs and then washed and expanded (PE) with irradiated uAPC and IL-2 (200 U/ml). NK cells were loaded with Amivantamab 1 μg/ml for one hour at 37ºC in Click's/RPMI media and were washed before validating the binding of Amivantamab to NK cells with flow cytometry. Non-loaded NE and PE NK cells were used as negative control. Amivantamab binding was detected by flow cytometry staining using Alexa-Fluor647 affinity-purified F(ab′)2fragment goat anti-human IgG (H+L) antibody. The graphs show that PE NK cells show higher level of Amivantamab binding compared to NE NK cells. Histograms show data from 3 different cord blood NK cell donors.

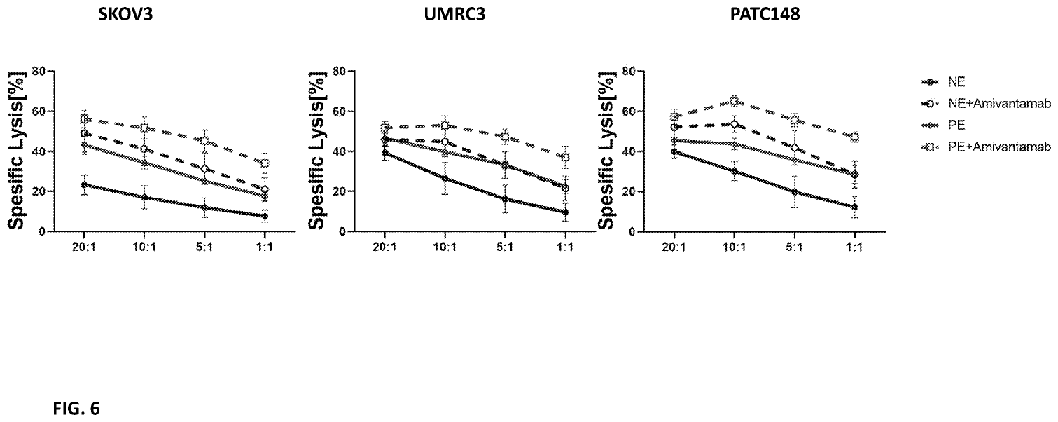

Amivantamab-loaded NK cells show enhanced cytotoxicity against EGFR+/cMET+tumor cells (FIG. 6). Chromium release assay showing specific lysis of 3 EGFR+/c-MET+ cancer cells lines SKOV3 (ovarian cancer), UMRC3 (renal cell carcinoma), PATC148 (pancreatic ductal adenocarcinoma) using 3 different cord blood NK cells (N=3 per assay) at various effector to target (E:T) ratios (20:1, 10:1, 5:1, 1:1) is provided. NK cells were either normally expanded (NE) or preactivated and expanded (P+E). NE NK cells were cultured with irradiated (100 Gy) uAPC feeder cells (2:1 feeder cell:NK ratio) and recombinant human IL-2 (200 U/ml) in 50:50 Click's/RPMI media and PE cells were preactivated with IL-12 (10 ng/ml), IL-15 (50 ng/ml), and IL-18 (50 ng/ml) for 16 hrs and then washed and expanded with irradiated uAPC and IL-2 (200 U/ml). NK cells were either left unloaded or loaded with Amivantamab 1 μg/ml for one hour at 37ºC in Click's/RPMI media and then washed before the assay. Amivantamab loading enhances the cytotoxicity of NE and PE NK cells against tumor cells.

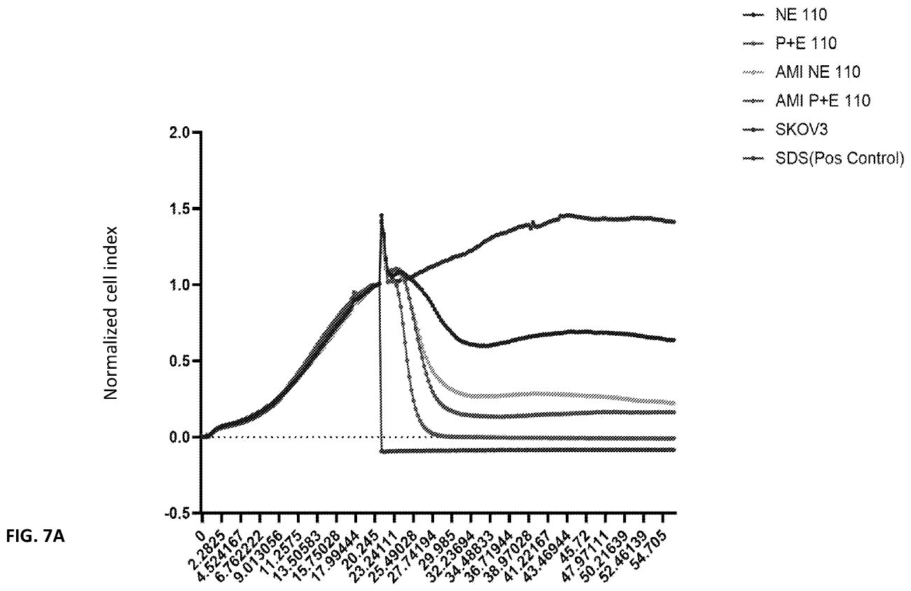

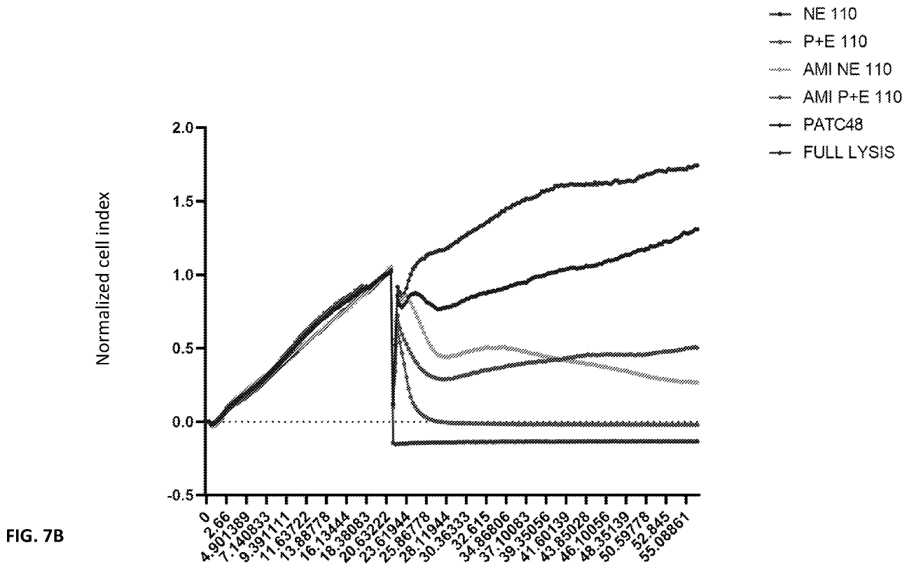

In FIGS. 7A and 7B, Amivantamab-loaded NK cells show enhanced cytotoxicity against EGFR+/cMET+tumor cells. Xcelligence cytotoxicity assay showing normalized cell index of 2 EGFR+/c-MET+ cancer cells lines SKOV3 (ovarian cancer; FIG. 7A) and PATC148 (pancreatic ductal adenocarcinoma; FIG. 7B) using 3 different cord blood NK cells (N=3 per assay) at 2:1 effector to target (E:T) ratio. NK cells were either normally expanded (NE) or preactivated and expanded (P+E). NE NK cells were cultured with irradiated (100 Gy) uAPC feeder cells (2:1 feeder cell:NK ratio) and recombinant human IL-2 (200 U/ml) in 50:50 Click's/RPMI media and PE cells were preactivated with IL-12 (10 ng/ml), IL-15 (50 ng/ml), and IL-18 (50 ng/ml) for 16 hrs and then washed and expanded with irradiated uAPC and IL-2 (200 U/ml). NK cells were either left unloaded or loaded with Amivantamab 1 μg/ml for one hour at 37° ° C. in Click's/RPMI media and then washed before the assay. Amivantamab loading enhances the cytotoxicity of NE and PE NK cells against tumor cells.

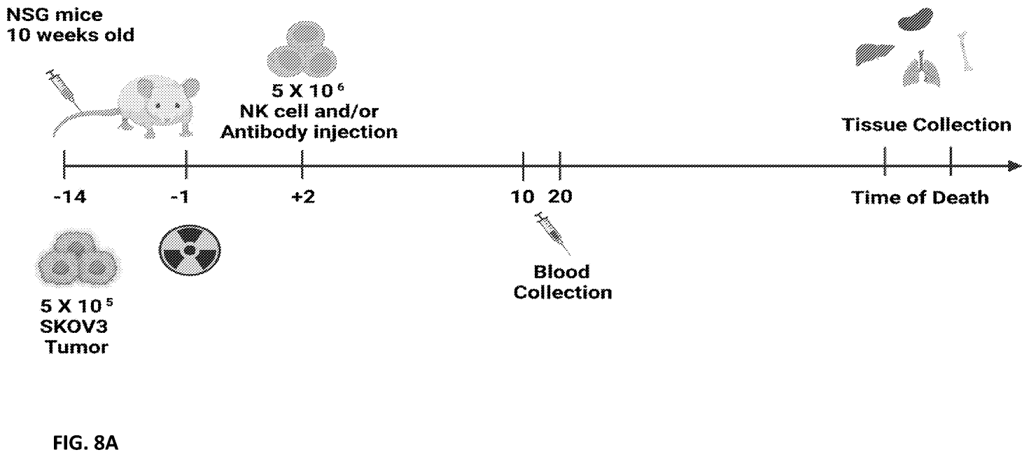

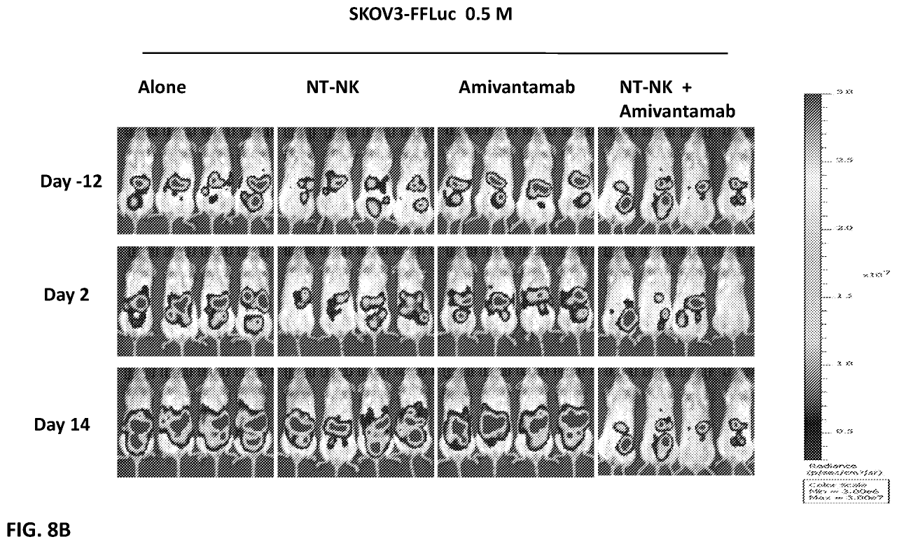

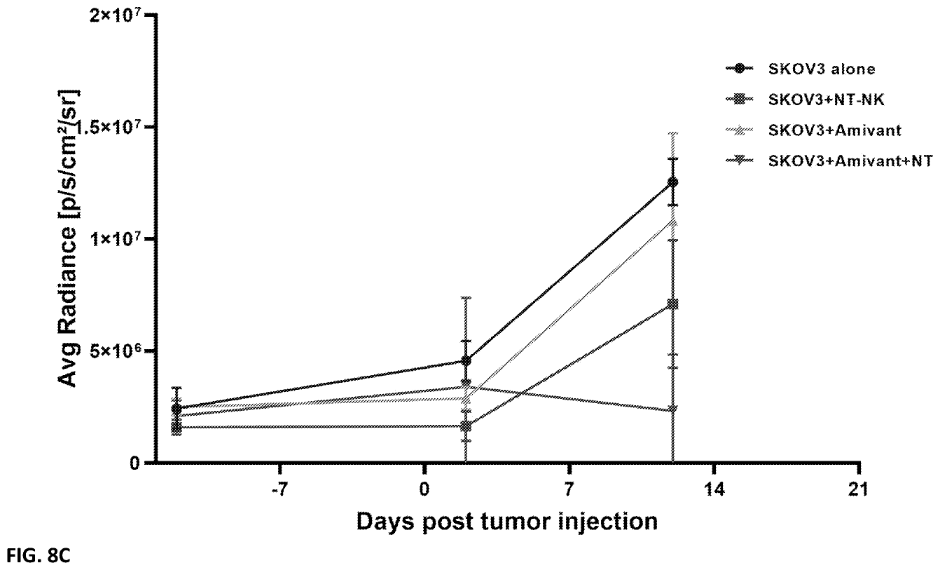

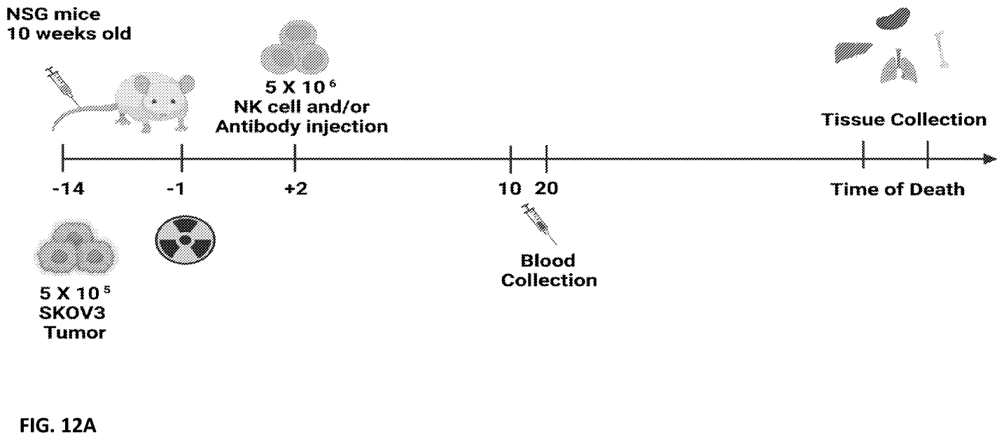

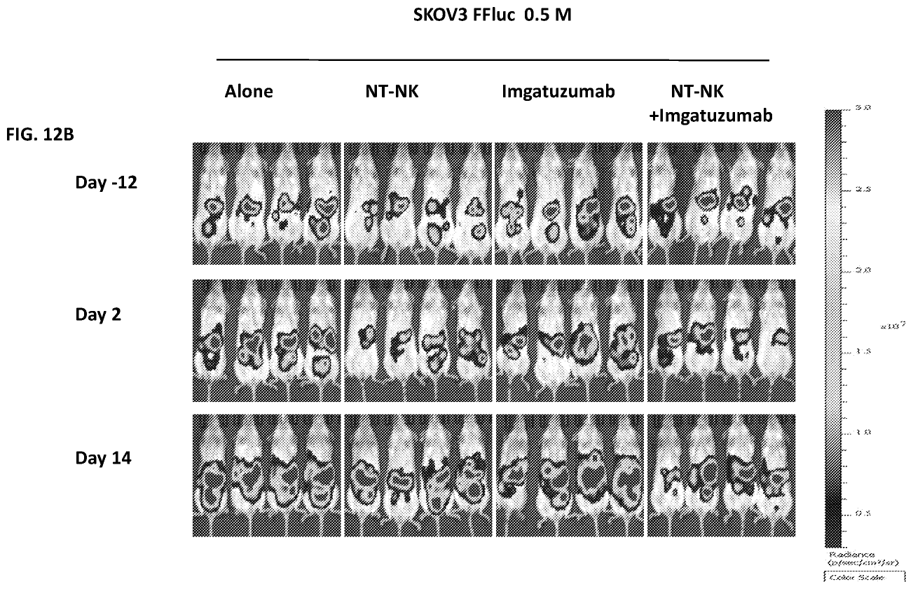

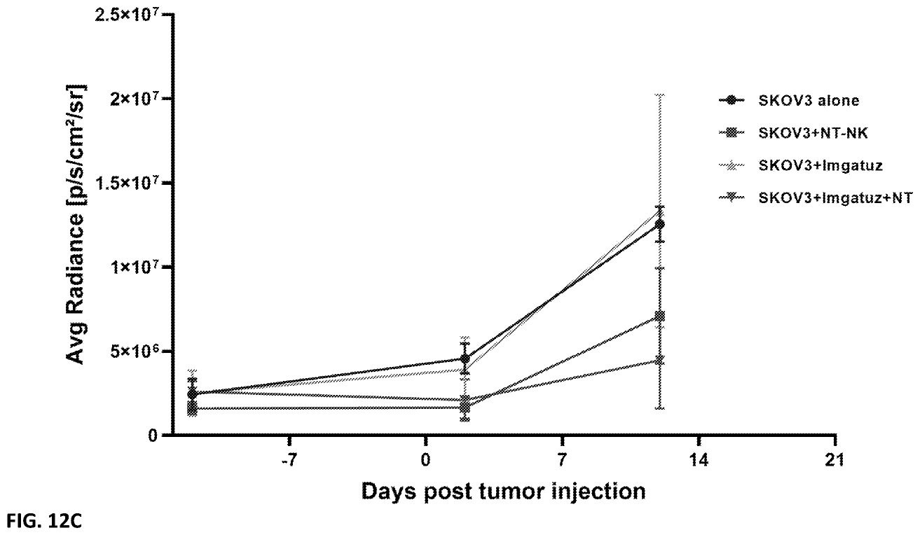

Amivantamab-loaded NK cells lead to enhanced tumor control in an NSG mouse model of EGFR+/c-MET+ovarian cancer (SKOV3) (FIG. 8). An example of a schema of experimental plan for SKOV3 mouse model is provided in FIG. 12A. In FIG. 8B, bioluminescent imaging shows tumor growth over time in mice engrafted with SKOV3 transduced with Fireflyluciferase (FFluc) and either left untreated or treated with NK cells alone, Amivantamab alone, or NK cells pre-loaded with Amivantamab. FIG. 8C provides a graph showing average radiance over time of the four groups of mice described in FIG. 8A.

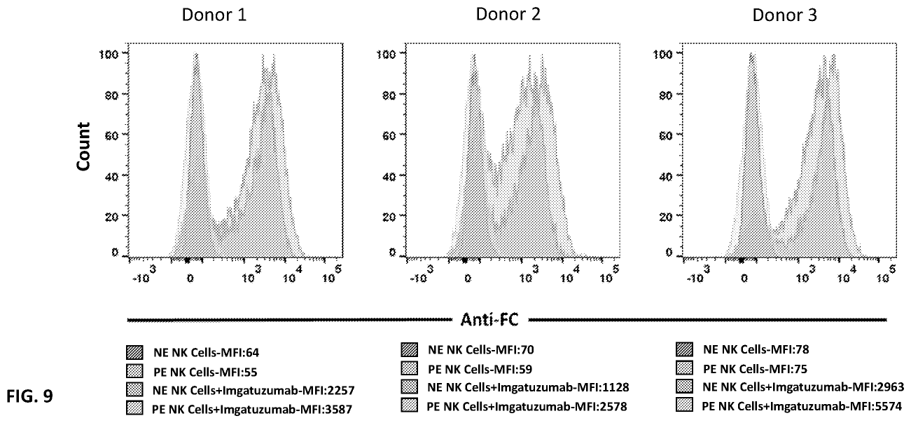

NK cells were loaded with one example of an antibody (FIG. 9), Imgatuzumab (an anti-EGFR monoclonal antibody). NK cells were derived from cord blood and were either normally expanded (NE) with irradiated (100 Gy) uAPC feeder cells (2:1 feeder cell:NK ratio) and recombinant human IL-2 (200 U/ml) in 50:50 Click's/RPMI media or pre-activated with IL-12 (10 ng/ml), IL-15 (50 ng/ml), and IL-18 (50 ng/ml) for 16 hrs and then washed and expanded (PE) with irradiated uAPC and IL-2 (200 U/ml). NK cells were loaded with Imgatuzumab at 1 μg/ml for one hour at 37ºC in Click's/RPMI media and were washed before validating the binding of Imgatuzumab to NK cells with flow cytometry. Non-loaded NE and PE NK cells were used as negative control. Imgatuzumab binding was detected by flow cytometry staining using Alexa-Fluor647 affinity-purified F(ab′)2fragment goat anti-human IgG (H+L) antibody. The graphs show that PE NK cells show higher level of Imgatuzumab binding compared to NE NK cells. Histograms show data from 3 different cord blood NK cell donors.

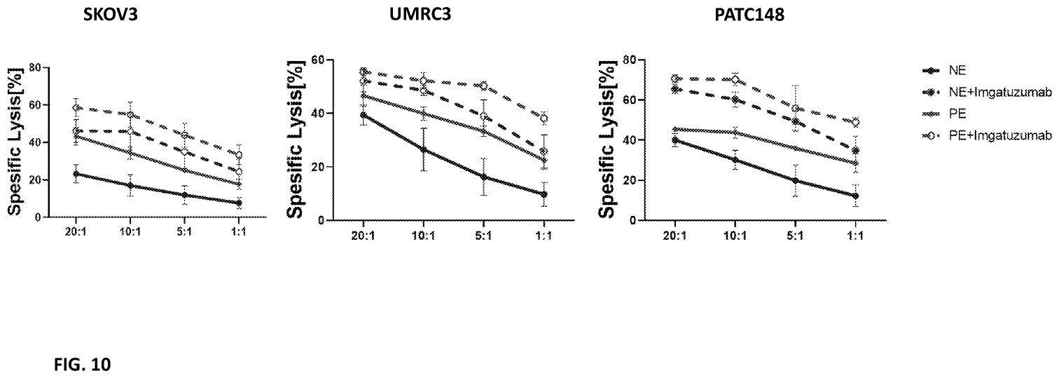

Imgatuzumab-loaded NK cells show enhanced cytotoxicity against tumor cells (FIG. 10). Chromium release assay showing specific lysis of 3 EGFR+ cancer cells lines (SKOV3 (ovarian cancer), UMRC3 (renal cell carcinoma), PATC148 (pancreatic ductal adenocarcinoma)) using 3 different cord blood NK cells (N=3 per assay) at various effector to target (E:T) ratios (20:1, 10:1, 5:1, 1:1). NK cells were either normally expanded (NE) or preactivated and expanded (P+E). NE NK cells were cultured with irradiated (100 Gy) uAPC feeder cells (2:1 feeder cell:NK ratio) and recombinant human IL-2 (200 U/ml) in 50:50 Click's/RPMI media and PE cells were preactivated with IL-12 (10 ng/ml), IL-15 (50 ng/ml), and IL-18 (50 ng/ml) for 16 hrs and then washed and expanded with irradiated uAPC and IL-2 (200 U/ml). NK cells were either left unloaded or loaded with Imgatuzumab 1 μg/ml for one hour at 37° C. in Click's/RPMI media and then washed before the assay. Data show that Imgatuzumab loading enhances the cytotoxicity of NE and PE NK cells against tumor cells.

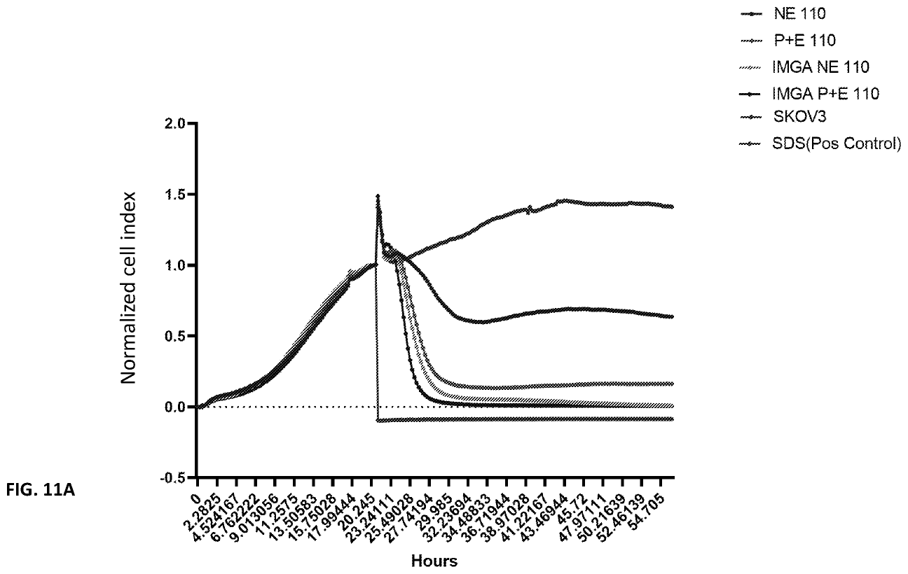

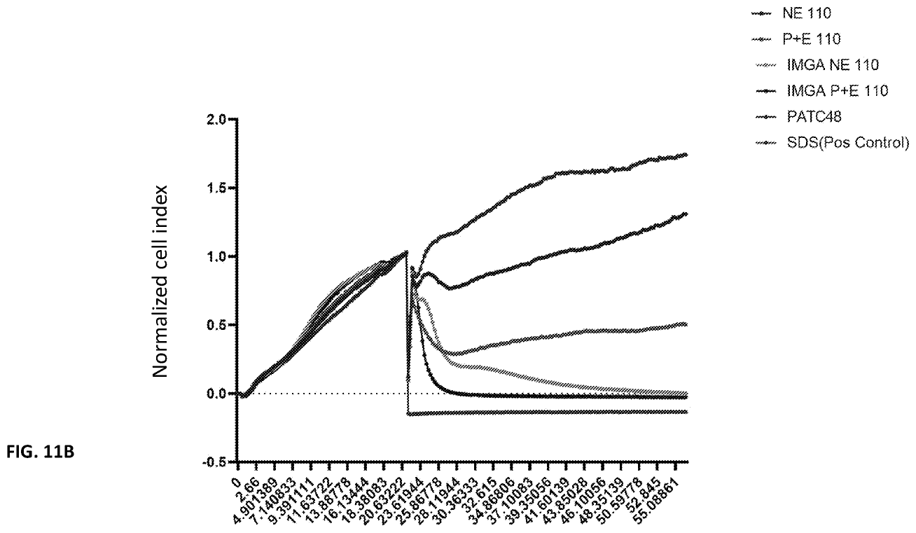

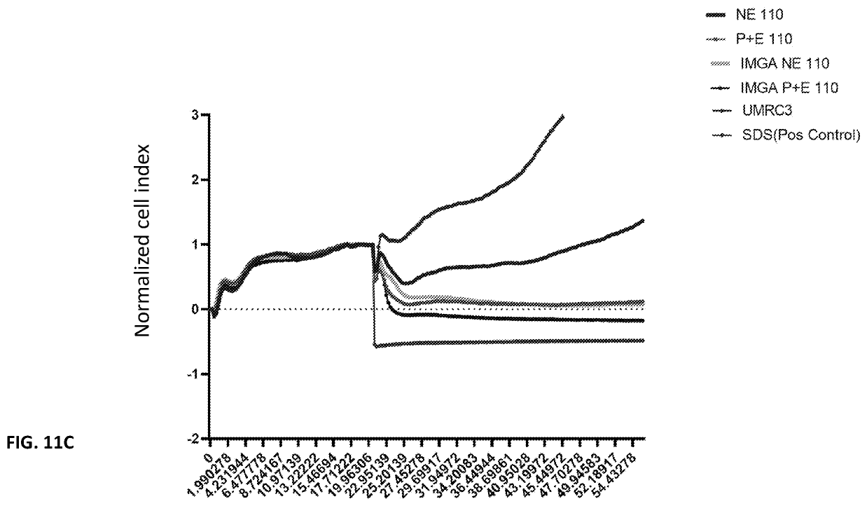

In FIGS. 11A-11C, Imgatuzumab-loaded NK cells show enhanced cytotoxicity against EGFR+tumor cells. The figure shows an Xcelligence cytotoxicity assay demonstrating normalized cell index of 3 EGFR+ cancer cells lines SKOV3 (ovarian cancer; FIG. 11A), PATC148 (pancreatic ductal adenocarcinoma; FIG. 11B), and UMRC3 (renal cell carcinoma; FIG. 11C) using 3 different cord blood NK cells (N=3 per assay) at 2:1 effector to target (E:T) ratio. NK cells were either normally expanded (NE) or preactivated and expanded (P+E). NE NK cells were cultured with irradiated (100 Gy) uAPC feeder cells (2:1 feeder cell:NK ratio) and recombinant human IL-2 (200 U/ml) in 50:50 Click's/RPMI media and PE cells were preactivated with IL-12 (10 ng/ml), IL-15 (50 ng/ml), and IL-18 (50 ng/ml) for 16 hrs and then washed and expanded with irradiated uAPC and IL-2 (200 U/ml). NK cells were either left unloaded or loaded with Imgatuzumab 1 μg/ml for one hour at 37° C. in Click's/RPMI media and then washed before the assay. Data indicate that Imgatuzumab loading enhances the cytotoxicity of NE and PE NK cells against tumor cells.

Imgatuzumab-loaded NK cells lead to enhanced tumor control in an NSG mouse model of EGFR+ovarian cancer (SKOV3) (FIG. 12). In FIG. 12A, there is one example of a schema of experimental plan for SKOV3 mouse model. FIG. 12B provides bioluminescent imaging showing tumor growth over time in mice engrafted with SKOV3 transduced with Fireflyluciferase (FFluc) and either left untreated or treated with NK cells alone, Imgatuzumab alone or NK cells pre-loaded with Imgatuzumab. In FIG. 12C there is a graph showing average radiance over time of the four groups of mice described in FIG. 12A.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A composition, comprising:
(1) one or more cord blood-derived natural killer (NK) cells; and
(2) one or more one antibody molecules, wherein:
(a) the antibody is monospecific, wherein an Fc region of the monospecific antibody binds the NK cell and an antigen binding domain of the monospecific antibody is capable of binding a target antigen; or
(b) the antibody is multispecific and one or more antigen binding domains of the antibody binds a target antigen and another antigen binding domain or domains of the antibody is capable of binding an NK cell surface antigen.

2. The composition of claim 1, wherein the NK cell is or is not expanded.

3. The composition of claim 1 or 2, wherein the NK cell is or is not pre-activated.

4. The composition of any one of claims 1-3, wherein the multispecific antibody is bispecific, trispecific, or multi-specific.

5. The composition of any one of claims 1-4, wherein in (a) the composition is further defined as a complex between the NK cell and the monospecific antibody, through binding of the Fc region of the monospecific antibody to the NK cell.

6. The composition of any one of claims 1-5, wherein the complex further comprises the antigen binding domain of the monospecific antibody bound to its target antigen.

7. The composition of any one of claims 1-4, wherein in (b) the composition is further defined as a complex between the NK cell and the multispecific antibody, through binding of the antigen binding domain or domains of the multispecific antibody that binds the NK cell surface antigen or antigens.

8. The composition of any one of claims 1-4 and 7, wherein the one or more antigen binding domain or domains of the antibody is bound to its target antigen.

9. The composition of any one of claims 1-6, wherein the target antigen is a stem cell antigen, auto-antigen, or a cancer antigen selected from the group consisting of CD19, CD319 (CS1), ROR1, CD20, CD22, CD70, carcinoembryonic antigen, alphafetoprotein, CA-125, MUC-1, Epidermal Growth Factor receptor (EGFR), epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD5, CD123, CD23, CD30, CD38, CD56, CD70, CD38, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, CD99, HLA-G, Trop2, and a combination thereof.

10. The composition of any one of claims 1-9, wherein the NK cell surface antigen is CD16, CS1, CD56, NKG2D, NKG2C, DNAM, 2B4, CD2, an NCR, or KIR.

11. The composition of any one of claims 1-9, wherein the source of the cord blood is cord blood from 1 donor or pooled from 2 or more individual cord blood units.

12. The composition of claim 11, wherein the CB is pooled from 3, 4, 5, 6, 7, or 8 individual cord blood units.

13. The composition of any one of claims 1-12, wherein the NK cells are derived from cord blood mononuclear cells or from cord blood hematopoietic stem cells.

14. The composition of any one of claims 1-13, wherein the NK cells are CD56+, CD3-, or both.

15. The composition of any one of claims 1-14, wherein said composition is used fresh or was cryopreserved.

16. The composition of any one of claims 1-15, wherein a source of the NK cells is a fresh source or cryopreserved repository.

17. The composition of claim 15 or 16, wherein when the NK cells are sourced from cryopreservation, the NK cells were cryopreserved in a medium comprising at least one cryoprotectant, at least one serum or non-serum alternative to serum, and optionally at least one cytokine and/or at least one growth factor.

18. The composition of claim 17, wherein the cryoprotectant is dimethyl sulfoxide (DMSO), glycerin, glycerol, hydroxyethol starch, dextran trehalose, or a combination thereof.

19. The composition of claim 17 or 18, wherein the non-serum alternative comprises platelet lysate and/or a blood product lysate or human or animal serum albumin.

20. The composition of any one of claims 17-19, wherein the at least one cytokine is a natural protein, a recombinant protein, a synthetic protein, or a mixture thereof.

21. The composition of any one of claims 17-20, wherein the at least one cytokine is interleukin (IL)-1, IL-2, IL-3, IL-4, IL-6, IL-7, IL-9, IL-10, IL-12, IL-13, IL-15, IL-17, IL-18, IL-21, IL-22, IL-23, interferon, tumor necrosis factor, stem cell factor, FLT3-ligand, APRIL, thrombopoietin, erythropoietin, or a combination thereof.

22. The composition of any one of claims 1-21, wherein the NK cells comprise one or more engineered antigen receptors.

23. The composition of claim 22, wherein the engineered antigen receptor is a chimeric antigen receptor, a T cell receptor, or both.

24. The composition of any one of claims 1-23, wherein the NK cells express a heterologous cytokine.

25. The composition of claim 24, wherein the heterologous cytokine is IL-2, IL-4, IL-7,IL-12, IL-15, IL-18, IL-21 or IL-23.

26. The composition of any one of claims 1-25, wherein the NK cells express one or more receptors to enhance their binding to an antibody.

27. The composition of claim 26, wherein the receptor is an Fc receptor.

28. The composition of claim 26, wherein the receptor is CD16, CD32, CD64, or a combination thereof.

29. The composition of any one of claims 1-28, wherein the NK cells express a suicide gene.

30. The composition of any one of claims 1-29, wherein the composition is comprised in a solution or solid comprising one or more cryoprotectants.

31. The composition of any one of claims 1-30, wherein the composition is comprised in a pharmaceutically acceptable carrier.

32. A method of producing the composition of any one of claims 1-31, comprising the steps of:
(a) optionally expanding NK cells in a culture comprising an effective amount of:
(1) a cytokine selected from the group consisting of IL-2, IL-15, IL-18, IL-21 and a combination thereof; and
(2) antigen presenting cells/feeders or NK activating beads; and
(b) providing the antibody molecules to the NK cells, and when expanding providing the antibody molecules to the NK cells before and/or after expanding.

33. The method of claim 32, wherein the method comprises a pre-activating step prior to and/or after the expanding step, wherein the NK cells are pre-activated in a culture comprising an effective concentration of one or more of IL-2, IL-12, IL-15, and IL-18.

34. The method of claim 33, wherein the culture comprises an effective concentration of two or more of IL-2, IL-12, IL-15, and IL-18.

35. The method of claim 33 or 34, wherein the culture comprises an effective concentration of three or more of IL-2, IL-12, IL-15, and IL-18.

36. The method of any one of claims 32-35, wherein the culture comprises an effective concentration of IL-12, IL-15, and IL-18.

37. The method of any one of claims 32-36, wherein IL-12 is utilized in lieu of IL-15 in the culture.

38. The method of any one of claims 32-37, wherein the providing step is further defined as culturing the NK cells with the antibody molecules for a specific duration of time or combining the NK cells and the antibody molecules just prior to infusion.

39. The method of claim 38, wherein the duration of time is about 5 minutes to about 24 hours or more.

40. The method of claim 38 or 39, wherein the culture comprises Plasma-Lyte A and/or human serum albumin.

41. The method of any one of claims 32-40, wherein following culture, the compositions are infused into a recipient subject without washing first.

42. The method of any one of claims 32-40, wherein following culture, the compositions are infused into a recipient subject following one or more washes.

43. The method of any one of claims 32-42, wherein the NK cells are depleted of CD3+, CD14+ and/or CD19+ cells.

44. The method of claim 43, wherein the depleting step occurs prior to the pre-activation step, and/or prior to expansion with feeder cells and/or NK cell-activating beads, and/or prior to culture with one or more cytokines, and/or prior to infusion.

45. The method of any one of claims 32-44, further comprising the step of obtaining the NK cells from cord blood, wherein the cord blood does not comprise cord tissue.

46. The method of any one of claims 32-45, wherein the antigen presenting cells are artificial (aAPCs).

47. The method of claim 46, wherein the aAPCs express CD137 ligand.

48. The method of claim 46 or 47, wherein the aAPCs further express a membrane-bound cytokine.

49. The method of claim 48, wherein the membrane-bound cytokine is membrane-bound IL-21 (mIL-21) or membrane-bound IL-15 (mIL-15).

50. The method of any one of claims 46-49, wherein the aAPCs have essentially no expression of endogenous HLA class I, II, or CD1d molecules.

51. The method of any one of claims 46-50, wherein the aAPCs express ICAM-1 (CD54) and/or LFA-3 (CD58) or CD48.

52. The method of any one of claims 46-51, wherein the aAPCs are further defined as leukemia cell-derived aAPCs.

53. The method of claim 52, wherein the leukemia-cell derived aAPCs are K562 cells engineered to express CD137 ligand and/or mIL-21.

54. The method of claim 53, wherein the K562 cells are engineered to express CD137 ligand and mIL-21.

55. The method of any one of claims 46-54, wherein the aAPCs have been engineered by retroviral transduction.

56. The method of any one of claims 46-55, wherein the aAPCs are irradiated.

57. The method of any one of claims 32-56, wherein the pre-activating step is for 10-20 hours.

58. The method of any one of claims 32-57, wherein the pre-activating step is for 14-18 hours.

59. The method of any one of claims 32-58, wherein the pre-activating step is for 16 hours.

60. The method of any one of claims 32-59, wherein the culture for the pre-activating step comprises IL-18 and/or IL-15 at a concentration of 1-1000 ng/ml.

61. The method of any one of claims 32-60, wherein the culture for the pre-activating step comprises IL-18 and/or IL-15 at a concentration of 1-1000 ng/mL.

62. The method of any one of claims 32-61, wherein the culture for the pre-activating step comprises IL-18 and/or Il-15 at a concentration of 1-1000 ng/mL.

63. The method of any one of claims 32-62, wherein the culture for the pre-activating step comprises IL-12 at a concentration of 0.1-1000 ng/mL.

64. The method of any one of claims 32-63, wherein the culture for the pre-activating step comprises IL-12 at a concentration of 1-1000 ng/mL.

65. The method of any one of claims 32-64, wherein the culture for the pre-activating step comprises IL-12 at a concentration of 10 ng/mL.

66. The method of any one of claims 32-65, further comprising washing the pre-activated NK cells prior to and/or after the expanding step.

67. The method of any one of claims 32-66, where NK cells are activated at least twice or more during the expansion step with IL-12, IL-15, IL-18, IL-2 or any combination thereof.

68. The method of claim 67, wherein washing is performed multiple times.

69. The method of any one of claims 34-68, wherein expanding is for 5-60 days.

70. The method of any one of claims 32-69, wherein the expanding is for 12-16 days.

71. The method of any one of claims 32-69, wherein the expanding is for 18-24 days.

72. The method of any one of claims 32-71, wherein the pre-activated NK cells and aAPCs are present in the expansion culture at a ratio of 3:1 to 1:3.

73. The method of claim 72, wherein the pre-activated NK cells and aAPCs are present in the expansion culture at a ratio 1:2.

74. The method of any one of claims 32-73, wherein the expansion culture further comprises IL-2.

75. The method of claim 74, wherein the IL-2 is present at a concentration of 10-500 U/mL.

76. The method of claim 75, wherein the IL-2 is present at a concentration of 100-300 U/mL.

77. The method of claim 76, wherein the IL-2 is present at a concentration of 200 U/mL.

78. The method of any one of claims 32-77, wherein the IL-12, IL-18, IL-15, and/or IL-2 are recombinant.

79. The method of any one of claims 32-78, wherein the IL-2 is replenished in the expansion culture every 2-3 days.

80. The method of any one of claims 32-79, wherein the APCs are added to the expansion culture at least a second time.

81. The method of any one of claims 32-80, wherein one or more steps of the method are performed in serum-free media.

82. A method of treating a disease or disorder in a subject, comprising administering a therapeutically effective amount of the compositions of any one of claims 1-31 to the subject.

83. The method of claim 82, wherein the disease or disorder is cancer, inflammation, graft versus host disease, transplant rejection, an autoimmune disorder, an immunodeficiency disease, a B cell malignancy, or an infection.

84. The method of claim 82 or 83, wherein the cancer is a hematological cancer or a solid tumor.

85. The method of claim 84, wherein the hematological cancer is a leukemia selected from the group consisting of an acute lymphoblastic leukemia (ALL), chronic lymphocytic leukemia (CLL), multiple myeloma, acute myelogenous leukemia (AML), and a chronic myelogenous leukemia (CML).

86. The method of any one of claims 82-85, wherein the NK cells are allogeneic with respect to the subject.

87. The method of any one of claims 82-86, wherein the NK cells are autologous with respect to the subject.

88. The method of claim 82, wherein the disorder is graft versus host disease (GVHD).

89. The method of claim 82, wherein the disorder is multiple sclerosis, inflammatory bowel disease, rheumatoid arthritis, type I diabetes, systemic lupus erythrematosus, contact hypersensitivity, asthma or Sjogren's syndrome.

90. The method of any one of claims 82-89, wherein the subject is a human.

91. The method of any one of claims 82-90, further comprising administering at least a second therapeutic agent to the subject.

92. The method of claim 91, wherein the at least a second therapeutic agent is a therapeutically effective amount of one or more anti-cancer agents, one or more immunomodulatory agents, and/or one or more immunosuppressive agents.

93. The method of claim 92, wherein the anti-cancer agent is chemotherapy, radiotherapy, gene therapy, surgery, hormonal therapy, anti-angiogenic therapy or immunotherapy.

94. The method of claim 92, wherein the immunosuppressive agent is a calcineurin inhibitor, an mTOR inhibitor, an antibody, a chemotherapeutic agent irradiation, a chemokine, an interleukins or an inhibitor of a chemokine or an interleukin.

95. The method of any one of claims 91-94, wherein the composition and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion.

96. The method of any one of claims 91-95, wherein the second therapeutic agent is an antibody.

97. The method of claim 96, wherein the antibody if a monoclonal, bispecific, or multi-specific antibody.
